(19) United States
(12) Patent Application Publication
(10) Pub. No.: US 2023/0340627 A1
(43) Pub. Date: Oct. 26, 2023

(54) ANALYSIS OF VIRAL PARTICLES BY DIGITAL ASSAY

(71) Applicant: Bio-Rad Laboratories, Inc., Hercules, CA (US)

(72) Inventors: Polina TSIGANKOV, Tirat Carmel (IL); Dalia SHEZIFI, Kibbutz Nahsholim (IL); Galina FIHMAN, Nesher (IL); Matt DUNN, Melrose, MA (US); Brigid CUNNION, Boulder, CO (US)

(21) Appl. No.: 18/296,357

(22) Filed: Apr. 5, 2023

Related U.S. Application Data
(60) Provisional application No. 63/327,727, filed on Apr. 5, 2022.

(51) Int. Cl. C12Q 1/70; G01N 33/569

(52) U.S. Cl. CPC C12Q 1/701; G01N 33/56983; C12Q 1/6851

(57) ABSTRACT

Methods, compositions, and kits for analyzing viral particles. In an exemplary method of analyzing viral particles, capsids of the viral particles may be tagged with a tag. Subsamples of a sample containing the viral particles may be formed. Each subsample of only a subset of the subsamples may include at least one of the viral particles. One or more targets may be amplified from a genome of the viral particles. Tag-related data, and amplification data for the one or more targets, may be collected from the subsamples. In some examples, a capsid occupancy of the viral particles may be determined in a calibration-free approach using the collected data without quantification of the viral particles or capsids thereof.

### CROSS-REFERENCE TO PRIORITY APPLICATION

This application is based upon and claims the benefit under 35 U.S.C. § 119(e) of U.S. Provisional Pat. Application Serial No. 63/327,727, filed Apr. 5, 2022. Each of these applications is incorporated herein by reference in its entirety for all purposes.

### INTRODUCTION

Adeno-associated virus (AAV) is a non-pathogenic human virus, and its recombinant form (rAAV) is used for gene therapy. During rAAV production, the desired products are full viral particles that contain a complete genome. However, defective viral particles having empty capsids (containing no genome) or partially-full capsids (containing a partial genome) are also generated as product-related impurities. These defective viral particles lack therapeutic benefit and may elicit unwanted immunotoxicity. Therefore, due to the clinical importance of capsid occupancy, the ratio of full capsids, partially-full capsids, and empty capsids in AAV gene therapy products is considered a Critical Quality Attribute (CQA).

Available methods to quantify this ratio are inadequate for various reasons, and can result in significantly different percent-full values for the same sample. No single “gold standard” method is currently recommended by regulatory agencies for quantifying capsid occupancy.

### SUMMARY

The present disclosure provides methods, compositions, and kits for analyzing viral particles. In an exemplary method of analyzing viral particles, capsids of the viral particles may be tagged with a tag. Subsamples of a sample containing the viral particles may be formed. Each subsample of only a subset of the subsamples may include at least one of the viral particles. One or more targets may be amplified from a genome of the viral particles. Tag-related data, and amplification data for the one or more targets, may be collected from the subsamples. In some examples, a capsid occupancy of the viral particles may be determined in a calibration-free approach using the collected data without quantification of the viral particles or capsids thereof.

### BRIEF DESCRIPTION OF THE DRAWINGS

FIG. 1 is a flowchart listing exemplary steps that may be performed in a method of analyzing viral particles.

FIG. 2 is another flowchart listing exemplary steps that may be performed in a method of analyzing viral particles.

FIG. 3 is a flow diagram schematically illustrating selected aspects of an exemplary method of analyzing viral particles for capsid occupancy by digital assay using a pair of proximity ligation assay (PLA) probes.

FIG. 4 is a schematic view of the pair of PLA probes and a connector from the flow diagram of FIG. 3, taken prior to annealing of the connector with the PLA probes and ligation of the PLA probes to one another to create a template.

FIG. 5 is another schematic view of the pair of PLA probes and the connector of FIG. 3, taken after creation of the template and illustrating amplification of a first target from the template in the presence of a target-specific amplification probe.

FIG. 6 is a schematic view of a viral genome from the flow diagram of FIG. 3 and illustrating amplification of a second target from the viral genome in the presence of a target-specific amplification probe.

FIG. 7 is a magnified, more detailed view of a scatter plot of collected amplification data from the flow diagram of FIG. 3.

FIG. 8 is a schematic view of a recombinant adeno-associated virus (“GFP-AAV2”) configured to express a green fluorescent protein (GFP) transgene.

FIG. 9 is a table listing capsid occupancy values determined by transmission electron microscopy (TEM) for various predefined ratios of “Full” GFP-AAV2 particles to “Empty” AAV2 particles.

FIG. 10 is a table showing a two-dimensional array of dilutions and full:empty ratios for comparing an exemplary digital assay of the present disclosure with the capsid occupancy values of FIG. 9 obtained by a different technique.

FIG. 11 is a table that is an expanded version of the table of FIG. 10, with cells of the table containing experimental values obtained using an exemplary digital assay of the present disclosure, TEM, or qPCR (Quantitative PCR)/ELISA (Enzyme-Linked Immunosorbent Assay).

FIG. 12 is series of tables presenting an exemplary calculation of capsid occupancy from experimental data obtained with the digital assay of FIG. 11.

FIG. 13 is a plot comparing different methods of calculating the percentage of capsids containing genomes (i.e., the % Full Capsids).

FIGS. 14 and 15 are plots showing copies/µL versus capsid concentration for PLA and GC (GFP) for various capsid occupancies.

FIG. 16 is a flow diagram schematically illustrating selected aspects of an exemplary method of analyzing viral particles for capsid occupancy by digital assay using beads and a tagging probe including a preformed, full-length template.

FIG. 17 is a flow diagram schematically illustrating selected aspects of an exemplary method of analyzing viral particles for capsid occupancy by digital assay using a labeled tagging probe to detect capsids.

FIG. 18 is a magnified view of the labeled tagging probe of FIG. 17.

### DETAILED DESCRIPTION

Various aspects and examples of methods, compositions, and kits for analysis of viral particles are described below and illustrated in the associated drawings. Unless otherwise specified, the methods, compositions, and kits may, but are not required to, contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein may be included in other similar methods, compositions, and kits, including being interchangeable between disclosed examples. The following description of various examples is merely illustrative in nature and is in no way intended to limit the examples, their applications, or their uses. Additionally, the advantages provided by the examples described below are illustrative in nature and not all examples provide the same advantages or the same degree of advantage.

The present disclosure provides methods, compositions, and kits for analyzing viral particles. In an exemplary method of analyzing viral particles, capsids of the viral particles may be tagged with a tag. Subsamples of a sample containing the viral particles may be formed. Each subsample of only a subset of the subsamples may include no viral particle, and each subset of another subset of subsamples may include at least one (or only one) of the viral particles. One or more targets may be amplified from a genome of the viral particles. Tag-related data, and amplification data for the one or more targets, may be collected from the subsamples. In some examples, a capsid occupancy of the viral particles may be determined in a calibration-free approach using the collected data without quantification of the viral particles or capsids thereof.

The data (tag-related data and amplification data) may be used to determine a value(s) for any suitable property or properties of the viral particles. In some examples, the data may be processed to separately quantify the capsids and the viral genome. However, since a specific binding agent, such as an antibody, may be used to enable capsid tagging for capsid detection, the capsid quantification is not absolute without calibration, because the tagging efficiency with specific binding agents is less than 100% and dependent on the affinity of the specific binding agents for their partners. Accordingly, capsid quantification may involve comparing the tag-related data, or a value(s) derived therefrom, to a calibration curve generated separately with known absolute amounts/concentrations of capsid. This comparison allows translation of the relative/approximate capsid quantification from tag-related data described above into an absolute amount/concentration for the capsids of the viral particles. In contrast, the viral genome can be quantified absolutely with sufficient accuracy from the amplification data without referring to a separate calibration curve, due to the inherently high efficiency of target amplification. With the capsids and the viral genome quantified absolutely (e.g., as concentrations) in this calibration-dependent approach, an accurate capsid occupancy (e.g., percent full capsids) can be calculated as a ratio based on the absolute viral genome concentration and the absolute concentration of the capsids.

The present disclosure also provides a calibration-free method of quantifying capsid occupancy without the need for a separate calibration curve. The calibration-free method can determine a value for capsid occupancy with sufficient accuracy without quantification of the capsids (or the viral particles). In this method, a ratio of subsample/partition counts from two or more populations of subsamples/partitions may be calculated to determine the capsid occupancy. The ratio utilizes relative amounts/concentrations of (i) viral particles including a capsid but not a copy (or at least not a complete copy) of the viral genome (i.e., the capsid is empty) and (ii) viral particles including both a capsid and a copy of the viral genome (i.e., the capsid is full). The same (typically unknown) tagging efficiency of the capsids, whether full or empty, is inherently present in the both the numerator and the denominator of the ratio and thus does not affect the ratio or need to be measured. If needed, the capsid concentration can then be calculated using the absolute quantification of the viral genome and the capsid occupancy value.

Further aspects of the present disclosure are described in the following sections: (I) definitions, (II) overview, (III) examples, components, and alternatives, (IV) illustrative combinations and additional examples, (V) advantages and benefits, and (VI) conclusion.

Features, functions, and advantages may be achieved independently in various examples of the present disclosure, or may be combined in yet other examples, further details of which can be seen with reference to the following description and drawings.

### I. Definitions

Technical terms used in this disclosure have meanings that are commonly recognized by those skilled in the art. However, the following terms may be further defined as follows.

An “amplicon” is a product of an amplification reaction. Copies of an amplicon may be generated by amplification of a target sequence, such that the amplicon corresponds to the target sequence (i.e., matches and/or is complementary to the target sequence). However, the sequence of the amplicon, such as at primer binding sites, may not exactly match and/or may not be perfectly complementary to the target sequence.

“Amplification” is a process whereby multiple copies are made of an amplicon matching, complementary, and/or otherwise corresponding to a target sequence. The process interchangeably may be called an amplification reaction. Amplification may generate an exponential increase in the number of copies as amplification proceeds. Typical amplifications may produce a greater than 100-fold or 1,000-fold increase in the number of copies of an amplicon. Exemplary amplification reactions for the methods disclosed herein may include a polymerase chain reaction (PCR) or a ligase chain reaction (LCR), each of which is driven by thermal cycling. The methods also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and/or the like. Amplification may utilize a linear or circular template.

“Amplification reagents” are any reagents that promote generation of an amplicon by amplification of a target sequence. The reagents may include any combination of at least one primer or primer pair for amplification of at least one target sequence, at least one label for detecting amplification of the at least one target sequence (e.g., at least one probe including a fluorophore and/or an intercalating dye as a label), at least one polymerase enzyme and/or ligase enzyme (which may be heat-stable), and nucleoside triphosphates (dNTPs and/or NTPs), among others.

A “capsid” (also called a “viral capsid”), as used herein, is a viral housing to contain and surround viral genetic material. Accordingly, a viral particle devoid of viral genetic material is a capsid (i.e., an empty capsid). The viral housing has a shell to enclose a viral genome. In most or all viruses, the shell is a proteinaceous shell composed of capsid proteins. In some cases, as with a typical non-enveloped virus, the viral housing is formed completely by the shell. In these cases, the shell provides an interior and an exterior of the viral housing. In other cases, as with enveloped viruses, the viral housing has additional components located outside the shell. The additional components may coat the shell and form one or more layers, such an envelope and a matrix, where the matrix is located between the shell and the envelope. The envelope includes a lipid bilayer and generally also includes proteins integral with, or otherwise associated with, the lipid bilayer. Any protein of the envelope may be virally-encoded or host-encoded.

Each capsid of a set of viral particles may be described as being “full” or “empty” with respect to a viral genome and/or one or more nucleic acid sequences thereof, which means, respectively, that the viral genome or sequence(s) is present or not present (or deemed to be present or not present) inside the capsid. The viral genome may be a native genome of a native virus, or a recombinant genome including a transgene or other non-native/engineered sequence. An “occupancy” of a set of viral particles (or capsids thereof) is a numerical description of a genome/target content of the viral particles (or capsids thereof). Exemplary units for capsid occupancy include a percent/fraction of capsids that are full, a percent/fraction of capsids that are only partially full, a percent/fraction of capsids that are empty, a ratio of full to empty capsids, or a ratio of empty to full capsids, among others.

A “carrier fluid” is a fluid that contacts partitions, optionally enclosing each partition. The fluid may be liquid or gas. The carrier fluid may be described as a continuous phase and the partitions therein as a dispersed phase. The carrier fluid may be immiscible with, and encapsulate each partition. In some examples, the carrier fluid may be an oil, such as including a fluorocarbon oil or a silicone oil.

“Complementary” means related by the rules of base pairing. A first nucleic acid polymer, or region thereof, is “complementary” to a second nucleic acid polymer if the first nucleic acid polymer or region is capable of hybridizing with the second nucleic acid polymer in an antiparallel fashion by forming a consecutive (uninterrupted) or nearly consecutive series of base pairs (e.g., at least 5, 6, 7, 8, 9, or 10 consecutive base pairs). The first nucleic acid polymer (or region thereof) is termed “perfectly complementary” to the second nucleic acid polymer if hybridization of the first nucleic acid (or region thereof) to the second nucleic acid polymer forms a consecutive series of base pairs using every nucleotide of the first nucleic acid polymer or region thereof. A “complement” of a first nucleic acid polymer or region thereof is a second nucleic acid polymer or region thereof that is perfectly complementary to the first nucleic acid polymer or region thereof. The “complementarity” between a first nucleic acid polymer (or region thereof) and a second nucleic acid polymer (or region thereof) refers to the number or percentage of base pairs that can be formed when the first nucleic acid polymer (or region thereof) is optimally aligned for hybridization in an antiparallel fashion with the second nucleic acid polymer (or region thereof). A first nucleic acid polymer or region thereof that is complementary to a second nucleic acid polymer or region thereof generally has a complementarity of at least 80%, 90%, 95%, or 100%.

“Comprising,” “including,” and “having” (and conjugations thereof) are used interchangeably to mean including but not necessarily limited to, and are open-ended terms not intended to exclude additional, unrecited elements or method steps.

A “connector” is an oligonucleotide(s) configured to base-pair with sequences of a pair of PLA probes. In some examples, this base-pairing may position a 5′-end and a 3′-end of the pair of PLA probes in apposition with one another, to enable ligation of the 5′-end and 3′-end to one another, to create a template. In other examples, this base-pairing may position a 5′-end and a 3′-end of the connector in apposition with one another, to enable ligation of the 5′-end and 3′-end to one another, to create a template, optionally by circularizing the connector.

“Coupled” means to be in such relation that the performance of one influences the performance of the other, may include being connected, either permanently or releasably, whether directly or indirectly through intervening components, and is not necessarily limited to physical connection(s).

A “digital assay” is an investigative procedure(s) capable of detecting single copies of an analyte in a set of subsamples or partitions, in which each subsample/partition of only a subset of the subsamples/partitions contains one or more copies of the analyte. A “digital amplification assay” is a digital assay that utilizes an amplification reaction(s) to facilitate detection of single copies of a target(s). A digital assay may be performed with any suitable number of subsamples/partitions that gives a statistically significant result, such as at least twenty, one-hundred, one-thousand, or ten-thousand, among others.

A “droplet” is a small volume of liquid encapsulated by an immiscible fluid (e.g., encapsulated by an immiscible liquid, which may form a continuous phase of an emulsion). The immiscible liquid may include oil and/or may be composed predominantly of oil. Droplets disclosed herein may, for example, have an average volume of less than about 500 nL, 100 nL, 10 nL, or 1 nL, among others.

An “enumeration value” is any value that results from enumerating a tag/target-defined subset of a set of subsamples/partitions in a digital assay. The enumeration value may, for example, represent a number of subsamples/partitions in the set of subsamples/partitions that are positive for the presence of a given tag/target or two or more given tags/targets, negative for the presence of a given tag/target or two or more given tags/targets, positive for only a specified subset of one or more tags/targets of a set of tags/targets, negative for only a specified subset of one or more tags/targets of a set of tags/targets, or the like.

“Exemplary” means “illustrative” or “serving as an example.” Similarly, the term “exemplify” (or “exemplified”) means “to illustrate by giving an example.” Neither term implies desirability or superiority.

“First,” “second,” “alpha,” “beta,” and similar terms are used to distinguish or identify various members of a group, or the like, in the order they are introduced in a particular context and are not intended to show serial or numerical limitation.

“Fluorescence” is optical radiation emitted in response to absorption of light. As used herein, fluorescence is intended to cover any form of photoluminescence, in which absorption of one or more photons promotes an electron to an excited state and leads to subsequent emission of a new photon, whether from a singlet state, a triplet state, or other state. The excited state produced by absorption may have any suitable lifetime.

A “fluorophore” is any atom, functional group, moiety, or substance capable of fluorescence.

A “genome” of a viral particle, or a set of viral particles, is the genetic material (nucleic acid, DNA and/or RNA) contained in a capsid of the viral particle or the set of viral particles. The genome may be a recombinant/engineered genome constructed at least partially by human activity or a natural genome. A set of viral particles may be described equivalently as including genomes, or as including full/partial copies of a genome.

A “label” is any detectable marker or identifier associated with a member, structure, or substance, such as associated with a primer, probe, amplicon, capsid, subsample, partition, or the like. The label may be associated covalently with the member, structure, or substance, such as a label that is conjugated to an oligonucleotide or antibody, or associated non-covalently (e.g., by intercalation, hydrogen bonding, electrostatic interaction, encapsulation, etc.). Exemplary labels include optical labels, radioactive labels, magnetic labels, electrical labels, epitopes, enzymes, antibodies, oligonucleotides, etc. Optical labels are detectable optically via their interaction with light. Exemplary optical labels that may be suitable include fluorophores and quenchers, among others.

“Labeling” is any action, process, or procedure that connects or associates a label to or with a specified structure, member, or substance. Labeling may produce a non-covalent connection or association, or a covalent attachment, of the label to the structure, member, or substance.

A “no particle control” (NPC) is a test or procedure performed in the absence of the viral particles of interest. A value(s) obtained from a no particle control can be used to adjust one or more values from a corresponding test or procedure performed in the presence of the viral particles of interest.

A “nucleic acid polymer” is a molecule or molecular duplex of any length composed of naturally-occurring nucleotides (e.g., where the polymer is DNA and/or RNA), or a compound produced synthetically that can hybridize with DNA or RNA in a sequence-specific manner analogous to that of two naturally occurring nucleic acids, e.g., can participate in Watson-Crick base pairing interactions. A nucleic acid polymer may be composed of any suitable number of nucleotides, such as at least about 5, 10, 100, or 1000, among others. The term “nucleic acid” means one or more nucleic acid polymers.

A nucleic acid polymer may have a natural or artificial structure, or a combination thereof. Nucleic acid polymers with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (G)) or a pyrimidine (such as cytosine (C), thymine (T), or uracil (U))). Nucleic acid polymers with an artificial structure are analogs of natural nucleic acids and may, for example, be created by changes to the pentose and/or phosphate groups of the natural backbone and/or to one or more nucleobases. Exemplary artificial nucleic acid polymers include glycol nucleic acids (GNAs), peptide nucleic acids (PNAs), locked nucleic acids (LNAs), threose nucleic acids (TNAs), xeno nucleic acids (XNA), and the like.

The sequence of a nucleic acid polymer is defined by the order in which nucleobases are arranged along the backbone. This sequence generally determines the ability of the nucleic acid polymer to hybridize with another nucleic acid by hydrogen bonding. In particular, adenine pairs with thymine (or uracil) and guanine pairs with cytosine.

An “oligonucleotide” is a relatively short and/or chemically synthesized nucleic acid polymer. The length of an oligonucleotide may, for example, be 3 to 1000 nucleotides, among others. In some cases, an oligonucleotide may be labeled with at least one label, which may be conjugated to any suitable structure of the oligonucleotide. The at least one label may include at least one fluorophore and thus may be a fluorescent label. Each label may be conjugated to the oligonucleotide at any suitable position, including a 5′-end, a 3′-end, or intermediate the 5′- and 3′-ends.

“Optical radiation” means electromagnetic radiation in the optical spectrum, namely, ultraviolet light, visible light, and/or infrared light. The term “light” without modification has the same meaning as optical radiation.

A “particle” is a small, discrete piece or volume of matter. A particle may be substantially solid (i.e., a solid-phase particle), liquid (i.e., a liquid-phase particle), or a combination thereof, among others. Exemplary particles include droplets, beads, biological cells, and viral particles. An exemplary dimension (e.g., length or diameter) of particles include less than 2000, 1000, 500, 200, or 100 micrometers, and/or at least 5, 10, or 20 nanometers, among others.

“Partitions” are discrete volumes of fluid (i.e., fluid volumes) that are isolated from one another (also called isolated volumes). Each partition of a set of partitions may contain a portion of the same sample. The partitions may be separated from one another by fluid (e.g., oil or air), a wall(s) of a device(s), or a combination thereof, among others. Accordingly, the partitions may be droplets of an emulsion, or volumes held by wells, chambers (e.g., nanochambers having a capacity of less than 1 µL), tubes (e.g., microtubes having a diameter of less than 1 mm), or microfluidic devices, among others. The partitions may be the same diameter as one another and/or may be composed of the same amount of fluid as one another.

A “partition count” (or a “subsample count”) is a value for the number of partitions (or subsamples) in a specified group, such as a population of partitions (or subsamples) having a specified tag/target content. A partition count or subsample count may be an enumeration value. The partition/subsample count may be determined by counting and/or calculating. The partition/subsample count (or a value therefor) may be “uncalibrated,” which means the count or value has not been adjusted by, read from, or otherwise derived using, a calibration curve for capsid quantification.

The term “positive” when used to indicate a tag/target content of a viral particle, partition, subsample, or a population of partitions/subsample indicates that the viral particle, partition/subsample, or each partition/subsample of the population contains (or at least appears and/or is deemed to contain) at least one copy of a given tag/target or of each tag/target of a given set of tags/targets. The term “negative” when used to indicate a target content of a viral particle, partition/subsample, or population of partitions/subsamples indicates that the viral particle, partition/subsample, or each partition/subsample of the population does not contain (or at least appears and/or is deemed not to contain) at least one copy of a given tag/target or of each tag/target of a given set of two or more tags/targets.

A “primer” is an oligonucleotide (DNA/RNA or an analog thereof) capable of serving as a point of initiation of template-directed nucleic acid synthesis or ligation under appropriate reaction conditions (e.g., in the presence of a template to which the primer anneals, nucleoside triphosphates, and an agent for polymerization (such as a DNA or RNA polymerase or ligase, or a reverse transcriptase), in an appropriate buffer and at a suitable temperature). The primer may have any suitable length, such as 5 to 500 nucleotides, among others. The primer may be a member of a “primer pair” or “primer set” including a “forward primer” and a “reverse primer” that define the ends of an amplicon generated in an amplification reaction. (The adjectives “forward” and “reverse” are arbitrary designations relative to one another.) The forward primer hybridizes with a complement of the 5′-end region of a template sequence to be amplified, and the reverse primer hybridizes with the 3′-end region of the template sequence. The term “primer binding site” refers to a portion of a template (or its complement) to which a primer anneals. The full sequence of the primer need not be perfectly complementary to the primer binding site, just sufficiently complementary to anneal under the conditions of the reaction. Accordingly, the primer may have a 3′-end region that is complementary to the primer binding site, and a 5′-end region that is not complementary to the primer binding site (and forms a “5′-tail”).

A “probe” is any substance configured to facilitate or enable testing, identification, and/or detection of an analyte, such as viral particles, viral capsids, a viral genome, a tag, and/or a target, among other analytes. The probe may, for example, be a tagging probe or an amplification probe. In some examples, either type of probe may include an oligonucleotide. A tagging probe is any probe including at least a portion of a tag. An amplification probe is configured to enable detection of the occurrence of an amplification reaction and/or formation of an amplicon by the amplification reaction. In some examples, the amplification probe may be a fluorescent probe including an oligonucleotide labeled with a fluorophore. The amplification probe may be configured to hybridize with at least a portion of an amplicon generated by amplification. The amplification probe may, for example, be a hydrolysis probe, a molecular beacon probe, a strand displacement probe, or a labeled primer, among others.

A “Proximity Assay” (PA) is a test or procedure in which a template is created in the presence of the viral particles being tested, in a proximity-dependent manner. Copies of the template are created when copies of separate parts of the template are in proximity to one another on individual viral particles through binding to those same individual viral particles. The template may be created from one or more oligonucleotides by any suitable reaction(s), such as ligation (in a Proximity Ligation Assay (PLA)) or extension (in a Proximity Extension Assay (PEA)). A PA probe is an oligonucleotide-containing probe including only a portion of the template to be created, or that is complementary to at least a portion of the template to be created. The PA probe may include a specific binding agent, such as for binding directly to a capsid, or for binding indirectly to the capsid via a capsid-specific binding agent. The oligonucleotide and the specific binding agent of the PA probe are conjugated covalently or connected otherwise to one another. In some examples, the PA probe may be a PLA probe for use in a Proximity Ligation Assay, or a PEA probe for use in a Proximity Extension Assay.

A “specific binding agent” is any molecule, complex, substance, or member that binds specifically to something else. Specific binding may be characterized by a dissociation constant of less than about 10⁻⁴, 10⁻⁶, 10⁻⁸, or 10⁻¹⁰ M. Exemplary pairs of specific binding agents include biotin and avidin/streptavidin, a sense nucleic acid and a complementary antisense nucleic acid (e.g., an amplification probe and an amplicon), a primer and its target, an antibody and a corresponding antigen, a receptor and its ligand, an aptamer and a corresponding antigen, and the like.

A “subsample” is a smaller sample provided by a bulk sample or sample-containing fluid and containing only a portion of the bulk sample or sample-containing fluid. A set of subsamples may be a set of partitions, and vice versa.

“Substantially” means to be predominantly conforming to the particular dimension, range, shape, concept, or other aspect modified by the term, such that a feature or component need not conform exactly, so long as it is suitable for its intended purpose or function. For example, a “substantially cylindrical” object means that the object resembles a cylinder, but may have one or more deviations from a true cylinder.

A “tag” is any identifier or marker that is, or will be, associated with a member, structure, substance, or complex. The tag may be conjugated covalently to the member, structure, substance, or complex, such as a tag that is conjugated to an antibody, or may be associated non-covalently and/or indirectly (e.g., by hydrogen bonding, electrostatic interaction, encapsulation, etc.). Exemplary tags include antibodies, antigens, aptamers, beads, epitopes, enzymes, nucleotide sequences, oligonucleotides, etc. A tag can include or be attached to a label, covalently or non-covalently, to make the tag detectable via the label.

“Tagging” is any process or set of processes that results in association of a tag with a specified member, structure, substance, or complex. The association may be a direct attachment (e.g., covalent attachment or direct binding) or an indirect connection.

A “target” (also called a “target sequence”) is a nucleic acid polymer sequence (DNA and/or RNA) of any suitable length that is amplified in an amplification reaction. Exemplary target sequences are about 20-1000 nucleotides, or about 30-500 nucleotides, among others.

A “template” is a nucleic acid polymer including a target sequence and/or a complement thereof.

### II. Overview

This section provides an overview of the methods, compositions, and kits described herein.

A method of analyzing viral particles is provided. In the method, capsids of the viral particles may be tagged with a tag. Subsamples of a sample containing the viral particles may be formed. Each subsample of only a subset of the subsamples may include at least one of the viral particles. One or more targets may be amplified from a genome of the viral particles. Tag-related data, and amplification data for the one or more targets, may be collected from the subsamples.

Another method of analyzing viral particles is provided. In the method, capsids of the viral particles may be tagged with a template. Partitions of a sample containing the viral particles may be formed, such that each partition of only a subset of the partitions includes one of the viral particles. A first target may be amplified from the template in the partitions, and one or more other targets may be amplified from a genome of the viral particles in the partitions. Amplification data for the first target and each target of the one or more other targets may be collected from the partitions.

A composition for analyzing viral particles is provided. The composition may comprise a partition of a bulk sample containing viral particles. The partition may contain a single viral particle of the viral particles. A capsid of the single viral particle may be tagged with a tag. The partition may include reagents sufficient for performing amplification of a target from a genome of the viral particles, if the genome is present in the partition.

### III. Examples, Components, and Alternatives

The following subsections, A to J, describe selected aspects of exemplary methods, compositions, and kits, for analyzing viral particles. The examples in these subsections are intended for illustration and should not be interpreted as limiting the entire scope of the present disclosure. Each subsection may include one or more distinct examples, and/or contextual or related information, function, structure, and/or processes.

### A. Methods of Analyzing Viral Particles for Capsid Occupancy by Digital Assay

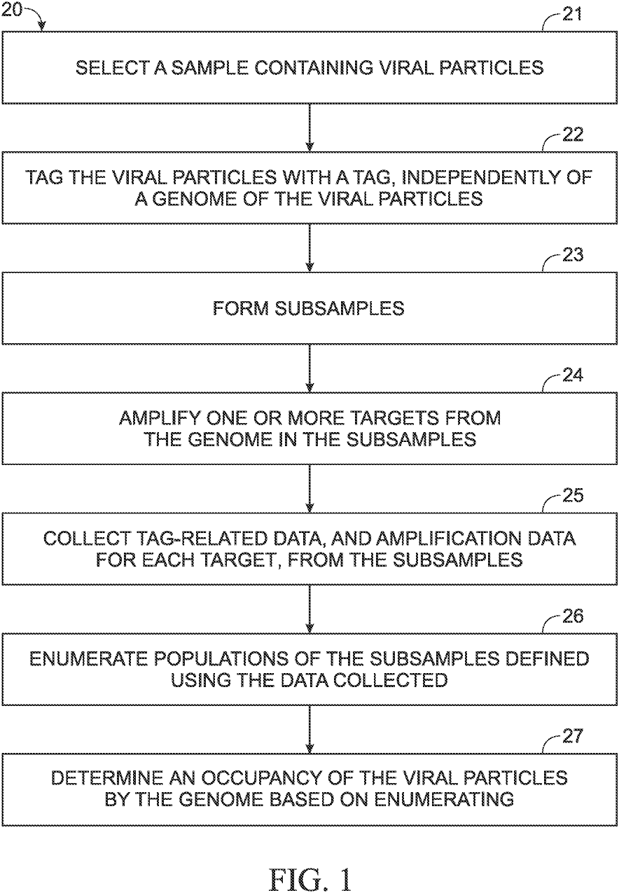

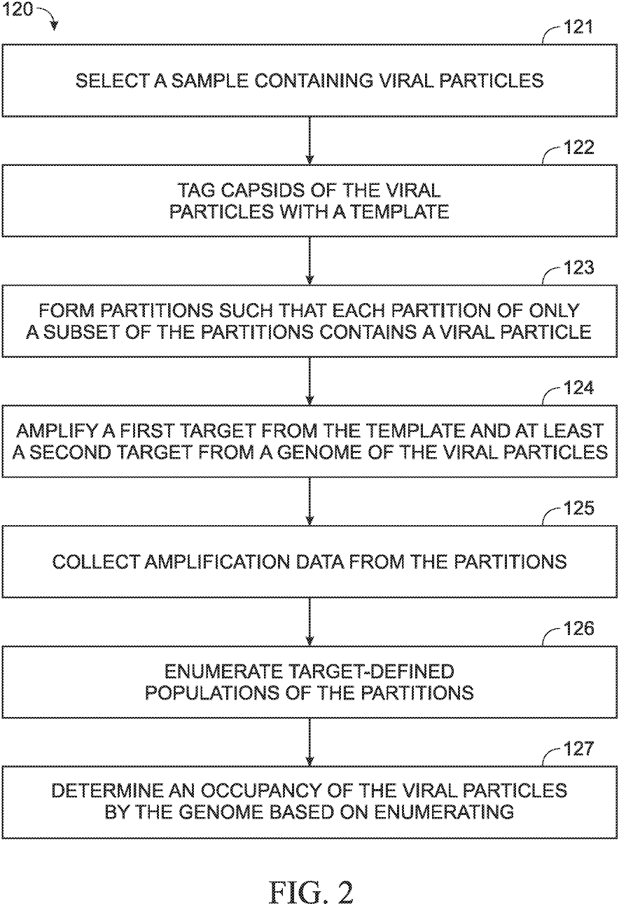

This subsection describes exemplary methods of analyzing viral particles by digital assay to quantify a capsid occupancy of the viral particles; see flowcharts 20, 120 of FIGS. 1 and 2. Each method may include any suitable combination of the steps listed in flowcharts 20 and 120, performed in any suitable order, and with any of the steps omitted or performed any suitable number of times, and using or including any of the features, aspects, reagents, configurations, additional steps, or modifications described elsewhere in the present disclosure.

In a selecting step 21 or 121 of flowcharts 20 and 120, a sample containing viral particles is selected (see FIGS. 1 and 2). The viral particles may include capsids (also described as copies of a capsid) and copies of a viral genome (also described as viral genomes), which are located inside the capsids. Each capsid may be a “full” capsid (or viral particle) containing a complete copy of the viral genome (and/or one or more predefined targets therein), a partially-filled capsid (or viral particle) containing only a portion of a copy of the viral genome (and/or only a subset (at least one, but less than all) of the one or more predefined targets), or an “empty capsid” (or viral particle) containing no copy of the viral genome (and/or none of the one or more predefined targets).

The viral particles may be a set of viral particles having structural similarity/identity to one another. At least a majority of the viral particles may have identical capsids and/or, for a majority of a subset of the viral particles containing a genome, identical copies of the genome.

The sample may be from any suitable source and may be prepared in any suitable manner. In some examples, the sample may be treated, such as with a nuclease (e.g., DNAse), to degrade unencapsidated copies of the viral genome and/or other nucleic acid located outside the viral particles. The nuclease then may be inactivated by addition of an inhibitor (e.g., EDTA), removal/inactivation of an activator or required cofactor, or by heating, among others.

The sample may contain any suitable type of viral particles. For example, illustrative viral particles may correspond to an adeno-associated virus, an adenovirus, a herpes virus, a lentivirus, a retrovirus, or the like. Accordingly, the viral genome may be composed of DNA or RNA, and the viral particles may or may not have respective viral envelopes surrounding the capsids.

In a tagging step 22 of flowchart 20, the viral particles are tagged with a tag (see FIG. 1). Tagging 22 is independent of a viral genome of the viral particles, meaning that individual viral particles are tagged whether or not they include a copy of the viral genome, generally with very similar or identical tagging efficiencies. The process of tagging may result in connection of the tag (at least one copy thereof) to each viral particle (and/or each capsid) of a plurality of the viral particles (and/or a plurality of the capsids thereof), for later colocalization of the viral particle (and/or capsid) and the at least one copy of the tag to the same subsample (and/or partition) (see below). The at least one copy of the tag may be connected covalently or non-covalently, and directly or indirectly, to the viral particle and/or capsid.

In some examples, it is not necessary that every one of the viral particles be tagged. For example, if a specific binding agent is involved in tagging, the efficiency of tagging may be significantly less than 100%, depending on the affinity of the specific binding agent. As described elsewhere herein, a characteristic of the viral particles, such as capsid occupancy, may be quantified in some examples, even though not all capsids are tagged, without knowing or measuring the tagging efficiency, and without a separate calibration to account for the tagging efficiency.

Tagging may include combining the viral particles (and/or the sample) and at least one tagging probe with one another. Each tagging probe may include a specific binding agent (e.g., an antibody, antibody fragment, or aptamer) and at least a portion of the tag. The specific binding agent and the at least a portion of the tag may be conjugated to one another covalently, or may be connected to one another non-covalently. In some examples, the specific binding agent itself may provide the tag.

The specific binding agent may have any suitable recognition and binding properties. In some examples, the specific binding agent may recognize and specifically bind directly to a structure of the viral particles that is separate from the viral genome (i.e., viral capsids, such as shells, matrices, or envelopes thereof). In other examples, the specific binding agent may bind to the structure of the viral particles indirectly via at least one other specific binding agent (e.g., a primary antibody), which in turn binds directly to the structure of the viral particles.

The tag may have any suitable properties to facilitate its detection. In some examples, the tag, or portions thereof, may include a detectable label (e.g., see Subsections I and J). In some examples, the tag may be configured to be connected to a detectable label during the method. In some examples, the tag may indirectly alter the detectability of a label.

In a tagging step 122 of flowchart 120, capsids of the viral particles are tagged with a tag providing a template (see FIG. 2). As described above with respect to step 22, the efficiency of tagging capsids is generally independent of capsid occupancy by a genome. Empty capsids and full capsids may be tagged with very similar or identical efficiencies. The process of tagging may result in connection of the tag/template (at least one copy thereof) to each viral particle (and/or each capsid) of a plurality of the viral particles (and/or a plurality of the capsids thereof), for later colocalization of the viral particle (and/or capsid) and the at least one copy of the tag/template to the same subsample (and/or partition) (see below). The tag/template may be created during tagging 122 in the presence of the viral particles, or may be combined with the viral particles as a preformed, full-length tag/template. In some examples, it is not necessary that every one of the viral particles be tagged, only a plurality and/or at least a subset of the viral particles, as explained above for tagging step 22. Accordingly, the efficiency of tagging may be significantly less than 100%.

Tagging 122 may include combining the viral particles (and/or the sample) and at least one tagging probe with one another. Each tagging probe may include a specific binding agent (e.g., an antibody, antibody fragment, and/or aptamer) and an oligonucleotide. The oligonucleotide provides at least a portion of the tag/template or is complementary to at least a portion of the tag/template (e.g., including a sequence of at least 5, 6, 7, 8, 9, or 10 nucleotides that matches or is perfectly complementary to a portion of the tag/template). The specific binding agent and the oligonucleotide of the tagging probe may be conjugated to one another covalently, or may be connected to one another non-covalently. In some cases, the oligonucleotide also may provide the specific binding agent.

Each tagging probe may be a Proximity Assay (PA) probe. Tagging 122 may include (i) combining the viral particles and a pair of PA probes (e.g., a pair of PLA probes or a pair of PEA probes) with one another, and (ii) creating the tag/template, such as by ligation (e.g., using a ligase) or by extension (e.g., using a polymerase).

If the pair of PA probes are PLA probes, at least one connector also may be combined with the viral particles before creating the tag/template. The at least one connector anneals to a pair of oligonucleotides of the pair of PLA probes to facilitate creation of the tag/template, such as by ligation of copies of the pair of oligonucleotides to one another. In some examples, copies of the pair of oligonucleotides are ligated to one another, and have different sequences relative to one another or are identical in sequence.

If the pair of PA probes are PEA probes, the PEA probes may provide a pair of oligonucleotides (a first oligonucleotide and a second oligonucleotide) configured to base-pair with one another at the 3′-end regions thereof. In other words, the first and second oligonucleotides may be complementary to one another at the 3′-end regions, which allows them to form a hybrid that can be extended by polymerase. More specifically, the 3′-end region of the first oligonucleotide may function as a primer that is extended using the second oligonucleotide as a template, and/or the 3′-end region of the second oligonucleotide may function as a primer that is extended using the first oligonucleotide as a template. In any event, extension of one or both of the oligonucleotides by the polymerase creates a tag/template that tags viral particles and/or capsids.

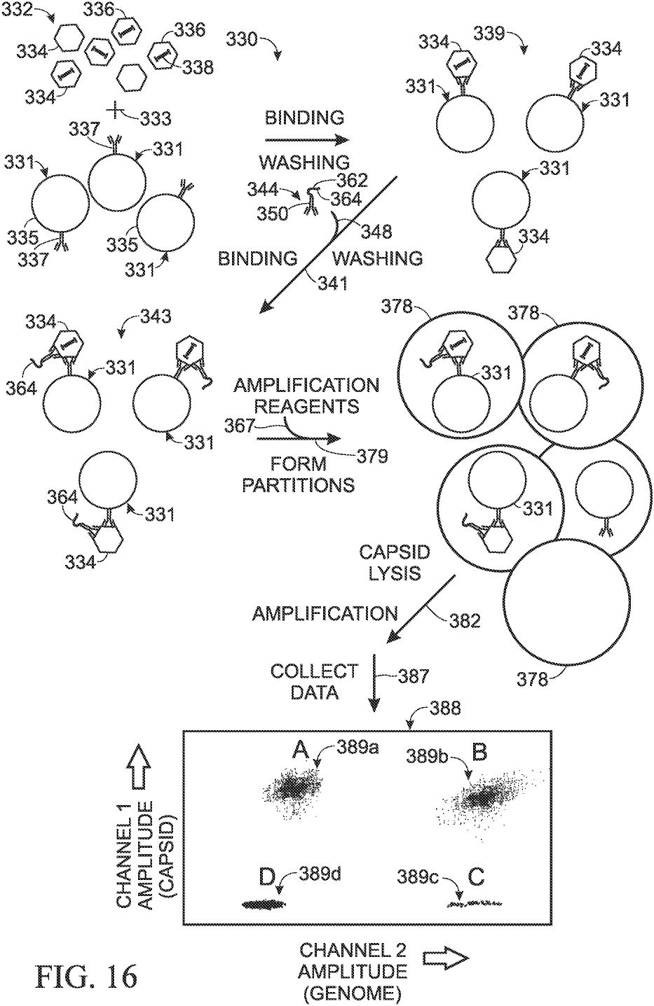

In other examples, tagging 122 may include combining the viral particles with a tagging probe that includes an oligonucleotide providing the full-length tag/template (also see Subsection H and FIG. 16). In this configuration, ligation may not be required.

Tagging 22 or 122 may be performed in the presence of beads (e.g., see Subsection H and FIG. 16). The viral particles may be combined advantageously with beads, such that the viral particles bind to the beads. The beads provide a solid support that may be washed after combining the beads and the viral particles with one another, and/or after combining the beads, viral particles, tagging probe(s), and/or connector(s) with one another, to remove viral particles that are not bound to the beads, and/or to remove copies of the tagging probe(s) and/or connector(s) that are not connected to the beads via viral particles. Accordingly, the use of beads and one or more washing steps may reduce background in any of the methods of the present disclosure.

In a forming step 23 of flowchart 20, subsamples are formed (see FIG. 1). The subsamples may be formed from a bulk sample containing the viral particles, such that each subsample contains a portion of the bulk sample. Only a subset of the subsamples contain a single viral particle, and/or only a subset of the subsamples contain at least one of the viral particles. The subsamples may have any combination of the properties described for partitions.

In a forming step 123 of flowchart 120, a set of partitions containing the viral particles are formed (see FIG. 2). Each partition of only a subset of the set of partitions may contain one or more of the viral particles, and thus another subset of the set of partitions may contain no viral particles. A plurality of partitions of the set of partitions each may contain only one viral particle. Stated differently, the set of partitions formed may contain a set of viral particles at “partial occupancy,” which means that the set of viral particles collectively is present in or contained by only a subset of the set of partitions. If the viral particles have been bound to beads (as described above) before forming step 123, the set of partitions also may contain the beads at partial occupancy. Viral particles of the set of viral particles (and the beads, if present) may be distributed randomly among the partitions, but in limited supply, such that every partition fails to receive at least one of the viral particles (and/or at least one of the beads). The partitions may be substantially uniform in size and may have any of the properties described above in Section J. In some examples, the partitions may be substantially identical to one another, except for differences in content of viral particles/beads and associated molecules. Each partition of the set of partitions may include a portion of the same sample-containing fluid, which, besides the sample, may contain amplification reagents (e.g., amplification primers, amplification probes, an amplification enzyme, etc.) and any other suitable reagents at a concentration/quantity sufficient for distribution of at least multiple copies of each reagent to every partition of the set of partitions.

Capsids may be lysed after forming subsamples/partitions to make copies of the viral genome accessible for amplification. Lysis is any alteration of the integrity of the capsids in the subsamples/partitions that releases viral nucleic acid from the capsids and/or allows amplification reagents to interact productively with the viral nucleic acid. The lysis may be driven by any suitable treatment that does not interfere with the integrity of the partitions, such as heating, ultrasonication, activation of a protease, exposure to detergent, and/or the like.

In an amplifying step 24 of flowchart 20, one or more targets are amplified from copies of a viral genome in the subsamples (see FIG. 1). The one or more targets may be amplified using a corresponding number of primers sets present in the subsamples. Amplifying in the subsamples may be performed at least generally as described below for partitions.

In an amplifying step 124 of flowchart 120, a first target may be amplified from the template and one or more other targets including a second target may be amplified from copies of the viral genome from the viral particles (see FIG. 2). The amplification is performed in the partitions and may be driven by heating the partitions above room temperature (e.g., to promote isothermal amplification) and/or thermally cycling the partitions (e.g., to promote a polymerase chain reaction or ligase chain reaction). In some examples, each partition may include amplification primers to amplify the first target and each of the one or more other targets, and an amplification probe specific for each target.

In a collecting step 25 of flowchart 20, tag-related data, and amplification data for each target, may be collected from the subsamples (see FIG. 1). The tag-related data may be used to detect the presence of the tag (and thus capsids/viral particles) in individual subsamples. The amplification data may be used to detect the presence of a viral genome in individual subsamples. Detecting from the subsamples may be performed at least generally as described below for partitions.

In a collecting step 125 of flowchart 120, amplification data may be collected from the set of partitions (see FIG. 2). The collecting step interchangeably is described as “reading” the partitions. In some examples, collecting amplification data may include detecting light, such as fluorescence, from the partitions. For example, the partitions may contain a target-specific amplification probe for each target, and fluorescence (e.g., fluorescence intensity) may be detected from a label (a fluorophore) of each target-specific amplification probe, optionally in a different detection channel for each label. Each detection channel may be defined with respect to the wavelength(s) of fluorescence detected, the wavelength(s) of excitation light, fluorescence lifetime, and/or the like.

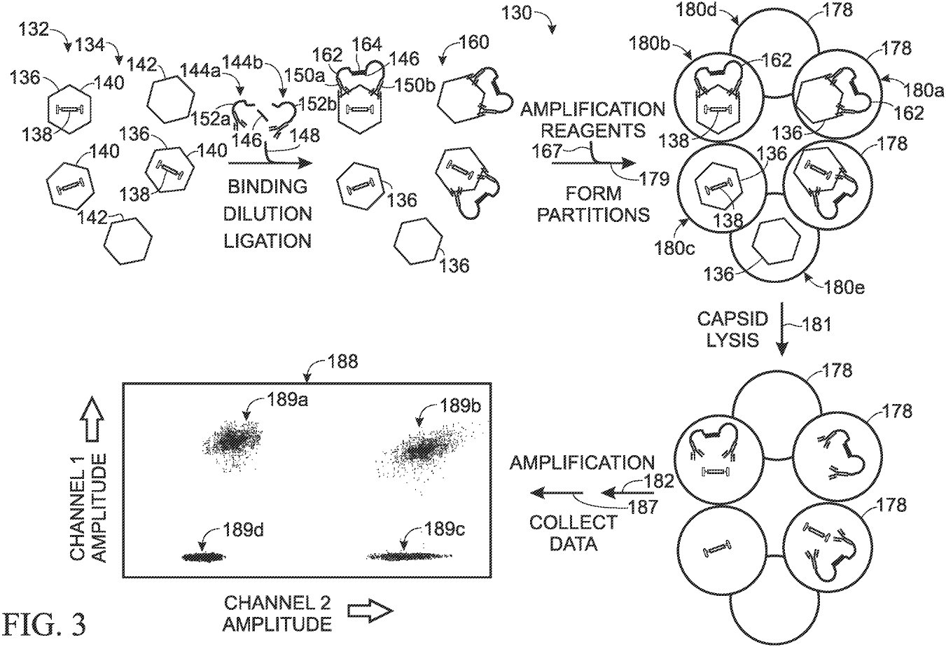

The tag-related data and/or the amplification data may be compared to one or more positivity/negativity thresholds to assign individual subsamples/partitions as positive or negative for the tag and/or each of the targets amplified. Each population of subsamples/partitions having the same tag/target content may be identified (e.g., see FIGS. 3, 7, 16, and 17). The tag/target content may be defined by positivity/negativity with respect to each tag/target.

In an enumerating step 26, 126 of flowcharts 20, 120, each of two or more populations of subsamples/partitions of the set of subsamples partitions may be enumerated to obtain a numerical value for a subsample/partition count from each of the two or more populations (see FIGS. 1 and 2). The numerical value represents the number of partitions present in the population. Each population has a different assigned tag/target content (positivity/negativity). Each subsample/partition count may be adjusted. Adjustment may include normalizing the subsample/partition count by scaling it up or down according to the ratio of a predefined or standard total set count of subsamples/partitions and the actual total set count of subsamples/partitions. For example, if the subsample/partition count for a given population is 100 from a total set count of 1000 subsamples/partitions assayed, the subsample/partition count normalized to a total set count of 2000 subsamples/partitions would be 100*(2000/1000) = 200. Adjustment also or alternatively may include subtracting a No Particle Control (NPC) value from the subsample/partition count (with or without normalization), to remove a background contribution to the subsample/partition count that is independent of viral particles.

In a determining step 27, 127 of flowcharts 20, 120, an occupancy of the viral particles by the genome is determined (see FIGS. 1 and 2). The occupancy may be a capsid occupancy of the viral particles by at least a portion of the viral genome. The capsid occupancy may be a numerical value that quantifies a fraction or percentage of the viral particles that contain a portion(s) of the viral genome corresponding to the one or more targets amplified from the viral genome.

### B. Homogeneous Digital Assay for Quantifying Capsid Occupancy

This subsection describes exemplary assay reagents for, and exemplary assay configurations produced by performance of, an illustrative method according to Subsection A, where the method includes a homogeneous digital assay for quantifying capsid occupancy and utilizes a pair of PLA probes to tag capsids; see FIGS. 3-7.

FIG. 3 shows a flow diagram 130 schematically illustrating selected aspects of the homogenous digital assay. A sample 132 containing viral particles 134 is shown at the top left of flow diagram 130. Viral particles 134 include viral capsids 136 enclosing copies of a viral genome 138. However, each capsid 136 does not enclose a copy of genome 138. Instead, viral particles 134 include full particles 140 containing a complete genome 138 in a capsid 136, and empty particles 142 lacking a genome 138 (or at least a portion thereof) in a capsid 136 and thus being defective. The goal of the homogeneous digital assay is to quantify occupancy in capsids 136 by genome 138 for viral particles 134.

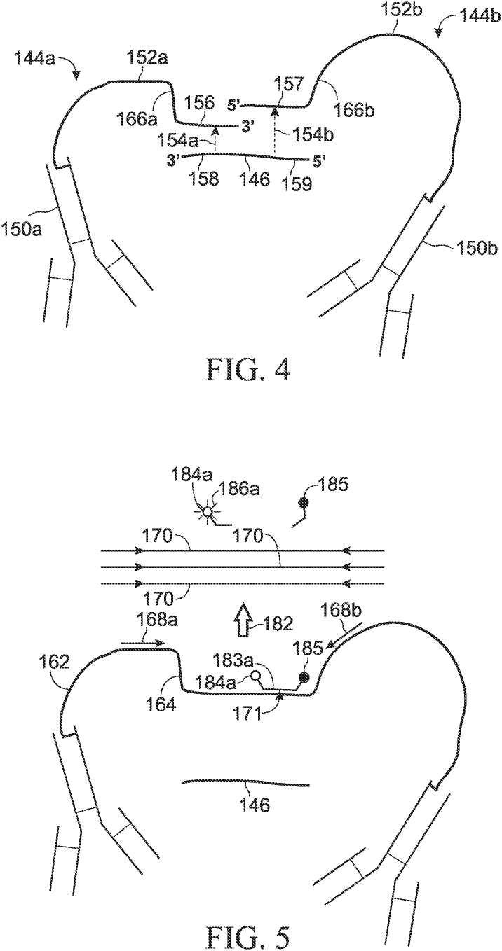

Sample 132 (with viral particles 134), a pair of tagging probes 144a, 144b, and a connector 146 are combined with one another, indicated at 148 (see FIGS. 3 and 4). In this example, tagging probes 144a, 144b are PLA probes and respectively include specific binding agents 150a, 150b to bind capsids 136 directly or indirectly, and oligonucleotides 152a, 152b for ligation to one another. The specific binding agents bind full capsids and empty capsids in a similar manner, with very similar or identical efficiencies. Each specific binding agent 150a, 150b includes an antibody, as shown here, although any type of specific binding agent may be suitable. If one or both specific binding agents 150a, 150b are configured to bind capsids 136 indirectly, combining may include combining one or more other specific binding agents with sample 132 and tagging probes 144a, 144b, to provide at least one bridge for connecting capsids 136 to one or both tagging probes 144a, 144b. Members of each pair of tagging probes 144a, 144b, specific binding agents 150a, 150b, and/or oligonucleotides 152a, 152b may be structurally different from one another or identical to one another.

FIG. 4 shows a magnified view of tagging probes 144a, 144b and connector 146 taken in the absence of a capsid 136 before ligation. Each specific binding agent 150a, 150b is conjugated to one of oligonucleotides 152a, 152b. In the depicted example, oligonucleotides 152a, 152b are conjugated respectively via a 5′-end and a 3′-end thereof to specific binding agents 150a, 150b. In other examples, each oligonucleotide may be conjugated, or attached non-covalently, to one of specific binding agents 150a, 150b at any position along the oligonucleotide.

Connector 146 is configured to base-pair, indicated by dashed arrows 154a, 154b, with a 3′-end region 156 of oligonucleotide 152a and a 5′-end region 157 of oligonucleotide 152b. In other words, a first sequence portion 158 of connector 146 is complementary to 3′-end region 156, and a second sequence portion 159 of connector 146 is complementary to 5′-end region 157. Hybridization of connector 146 with both 3′-end region 156 and 5′-end region 157 arranges the 3′-end and the 5′-end of oligonucleotides 152a, 152b in apposition to one another, to enable efficient ligation at the ends. In the depicted example, connector 146 is provided by an oligonucleotide that is separate from oligonucleotides 152a, 152b. In other examples, connector 146 may be provided by a connector sequence present in one of oligonucleotides 152a, 152b.

Combining 148 creates a mixture including viral particles 134, tagging probes 144a, 144b, and connector 146 (see FIG. 3), and may be performed in any suitable order. For example, viral particles 134 and tagging probes 144a, 144b may be combined with one another to create a first mixture. The first mixture may be incubated at a controlled temperature to permit binding of the tagging probes to capsids 136 and then diluted, if needed. The first mixture then may be combined with a DNA ligase and connector 146 to produce a ligation mixture 160 in which connector 146 hybridizes with copies of oligonucleotides 152a, 152b to enable ligation. The ligation mixture is incubated to permit ligation of tagging probes 144a, 144b to one another to create a tag 162 that tags capsids 136 (see FIGS. 3-5). More specifically, copies of oligonucleotides 152a, 152b of tagging probes 144a, 144b are ligated to one another to create tag 162, which provides a template 164 (see FIGS. 4 and 5). Template 164 includes respective template portions 166a, 166b from oligonucleotides 152a, 152b. However, the tagging efficiency is not 100%, such that some of capsids 136 are not tagged with template 164, as shown in ligation mixture 160 of FIG. 3.

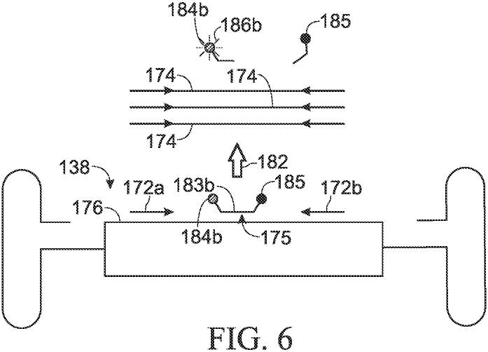

Ligation mixture 160 is then combined with amplification reagents, indicated at 167 (see FIG. 3). The amplification reagents include a pair of first primers 168a, 168b to amplify a first target 170 from template 164, and a first amplification probe 171 for detecting amplification of first target 170 (see FIG. 5). The amplification reagents also include a pair of second primers 172a, 172b for amplification of a second target 174 from genome 138, and a second amplification probe 175 for detecting amplification of second target 174 (see FIG. 6). In the depicted example, only a single pair of primers 172a, 172b are used to amplify one target from a transgene 176 of genome 138. In other examples, one or more additional primer sets (and probe(s)) may be used to amplify one or more other targets from genome 138, to better distinguish full viral particles from other viral particles lacking at least part of the genome.

After combining 167, a set of partitions 178 are formed, indicated at 179 (see FIG. 3). Partitions 178 include capsid+tag partitions (configuration 180a, containing a capsid 136 and tag 162, but no genome 138), capsid+tag+genome partitions (configuration 180b, containing a capsid 136, a genome 138, and tag 162), particle-only partitions (configuration 180c, containing a capsid 136 and a genome 138, but no tag 162), and target-negative partitions (configurations 180d and 180e), each containing no genome 138 or tag 162).

Partitions 178 are then treated (e.g., heated) to encourage capsid lysis, indicated at 181. Capsid lysis allows the amplification reagents and copies of genome 138 to contact one another for subsequent amplification, and may result in melting of nucleic acid duplexes to single-stranded form.

Amplification of each target is then performed in the partitions, indicated at 182 (see FIGS. 3, 5, and 6). Target amplification hydrolyzes an oligonucleotide 183a (first target) or an oligonucleotide 183b (second target) of amplification probes 171, 175, which separates labels, fluorophores 184a, 184b, of the probes from a quencher 185, such that fluorescence 186a, 186b increases (see FIGS. 5 and 6).

Amplification data is collected, indicated at 187, by detecting fluorescence, if any, from each partition in at least two detection channels (channel 1 and channel 2) (see FIG. 3). Fluorescence 186a from fluorophore 184a of first amplification probe 171, which represents amplification of first target 170, is detected in channel 1 (also see FIG. 5). Fluorescence 186b from fluorophore 184b of second amplification probe 175, which represents amplification of second target 174, is detected in channel 2 (also see FIG. 6).

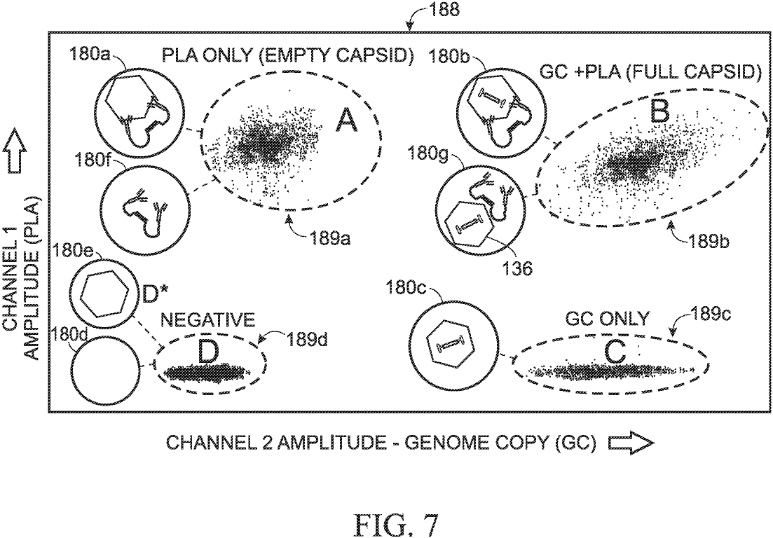

A scatter plot 188 of the amplification data uses a respective dot to represent each partition, and positions the dot within the plot according to the amplitude (e.g., intensity) of channel 1 and channel 2 fluorescence detected from the corresponding partition (see FIGS. 3 and 7). The dots/partitions are clustered in four populations 189a-189d (respectively also identified as “A”, “B”, “C”, and “D” in FIG. 7). Each partition can be assigned to one of the populations by comparing the amplitude of the partition in each detection channel (1 and 2) to a respective threshold.

Partitions 178 present in each population 189a-189d are superimposed on scatter plot 188 in FIG. 7. The partitions are shown in pre-lysis configurations, including configurations 180a-180e described above. Additional partition configurations 180f and 180g present in populations 189a and 189b, respectively, are also identified. Configurations 180f and 180g are positive for the first target, independent of the presence/absence of a capsid 136. In other words, ligation of tagging probes 144a, 144b to create template 164 occurs in the absence of a capsid 136 (configuration 180f) or without binding of one or both tagging probes 144a, 144b to a capsid 136 (configuration 180g) (also see FIGS. 3-5).

### C1. Quantification of Capsid Occupancy Using Partition Population Counts

This subsection provides an overview of a first method for quantifying capsid occupancy, specifically, using partition population counts. The subsection presents illustrative equations and calculations, and refers to partition counts A-D and D* (from populations 189a-189e, respectively) and partition configurations 180a-180g of Subsection B (see FIGS. 3-7).

Equation 1 states that the percent capsid occupancy (% full capsids) is equal to a ratio of the sum of partition counts for populations B and C (i.e., each partition containing the second target) over the sum of partition counts for populations A, B, C, and D* (i.e., each partition containing either or both of the first and second targets), multiplied by 100:

$$\% F u l l C a p s i d s = \frac{B + C}{A + B + C + D *} \times 100 \qquad \text{(1)}$$

D is negative for both the first and second targets. D* is a subset of D containing an empty capsid that is not labeled in the assay (see configuration 180e of FIG. 7). However, a partition count value for D* can be calculated from the capsid tagging efficiency (i.e., the “PLA efficiency”).

Equation 2 states that the PLA efficiency is equal to a ratio of the sum of partition counts for populations A and B (i.e., each partition positive for the first target) over the sum of partition counts for populations A, B, C, and D* (i.e., each partition containing either or both of the first and second targets), multiplied by 100:

$$P L A E f f i c i e n c y = \frac{A + B}{A + B + C + D *} \times 100 \qquad \text{(2)}$$

The PLA efficiency is assumed to be independent of the presence or absence of the second target. This assumption has been demonstrated to be valid empirically by the inventors. With this assumption, the PLA efficiency should be the same for populations A, B, C, and D* collectively, only the two populations, A and D*, negative for the second target, and only the two populations, B and C, positive for the second target, as shown in Equation 3:

$$\frac{A + B}{A + B + C + D *} = \frac{A}{A + D *} = \frac{B}{B + C} \qquad \text{(3)}$$

The middle and right-hand ratios of Equation 3 can used to solve for D* as given by Equation 4:

$$D * = \frac{A C}{B} \qquad \text{(4)}$$

Substituting for D* in Equation 1 based on Equation 4, and simplifying gives Equation 5:

$$\% F u l l C a p s i d s = \frac{B}{A + B} \times 100 \qquad \text{(5)}$$

Populations A and B each include a respective portion of a background signal produced by partitions in which spontaneous, capsid-independent generation of the first target template occurred (see configurations 180f and 180g of FIG. 7). The respective portions of the background signal for populations A and B may be subtracted to give A_{sub} and B_{sub} for calculating capsid occupancy according to Equation 6:

$$\% F u l l C a p s i d s = \frac{{B}_{s u b}}{{A}_{s u b} + {B}_{s u b}} \times 100 \qquad \text{(6)}$$

The background signal is measured in a No Particle Control (NPC) assay, which is performed with the same reagents and conditions, except using a control (NPC) sample that does not contain any of the viral particles. The background signal is measured from population A produced by the NPC sample (when no viral particles are present, population B is not generated). However, in a viral-containing sample, this background signal is divided between populations A and B, and therefore respective portions of the background signal should be subtracted from the two populations.

Each time the assay is performed, with either a viral-containing sample or an NPC sample, the assay can produce a different total number of accepted partitions (the total partition count). For example, in some embodiments, the total partition count may vary between 12,000 and 20,000. To correctly subtract the NPC background signal from populations A and B generated with a viral-containing sample, the partition count of each population (for a viral-containing sample(s) and an NPC sample(s)) may be normalized with respect to the same value for total partition count (e.g., 20,000). For example, for population A of assay “x”, the normalization of the partition count for Aₓ to A_{N}, which is based on a total partition count of 20,000, is given by Equation 7:

$${A}_{N} = \frac{{A}_{x} \times 20 , 000}{A c c e p t e d P a r t i t i o n {s}_{x}} \qquad \text{(7)}$$

In samples containing the viral particles, the background signal is unevenly distributed between populations A and B, in a manner dependent on the relative pool sizes of genome-negative partitions (A+D) and genome-positive partitions (B+C).

The fraction of background signal that is distributed to population A can be calculated using Equation 8:

$${D}_{f r} = \frac{D}{C + D} \qquad \text{(8)}$$

The fraction of background signal that is distributed to population B can be calculated using Equation 9:

$${C}_{f r} = \frac{C}{C + D} \qquad \text{(9)}$$

A normalized average NPC population A, A_{NPC}, is calculated when several NPC samples are tested. The respective NPC fraction is subtracted from normalized populations A and B of the viral-containing sample according to Equations 10 and 11, respectively:

$${A}_{s u b} = {A}_{N} - \left({A}_{N P C} \times {D}_{f r}\right) \qquad \text{(10)}$$

$${B}_{s u b} = {B}_{N} - \left({A}_{N P C} \times {C}_{f r}\right) \qquad \text{(11)}$$

### C2. Quantification of Capsid Occupancy Using Partition Population Counts and Linkage Concentrations

This subsection provides an overview of a second method for quantifying capsid occupancy, specifically, using partition population counts and linkage concentrations. The subsection presents illustrative equations, like the previous subsection, and refers directly or indirectly to partition counts A-D and D* (from populations 189a-189e, respectively) and partition configurations 180a-180g of Subsection B (see FIGS. 3-7). However, here population 180g includes partitions that have BOTH capsids 136, with genomes 138, that are not labeled with tags 162 (e.g., as shown in configuration 180c) AND capsids 136, without genomes 138, that are labeled with tags 162 (e.g., as shown in configuration 180a). In this case, the expression for % Full Capsids can be written in terms of partition population count and linkage concentrations as follows:

$$\% F u l l C a p s i d s = \frac{{B}_{L i n k}}{{\left(A + B\right)}_{s u b}} \times 100 \qquad \text{(12)}$$

Here, B_{Link} is the linkage concentration of double positive partitions (i.e., partitions positive for capsids and genomes), and (A + B)_{sub} is the concentration of labeled capsids after NPC subtraction. Significantly, Equation 12 may provide improved accuracy over Equation 6, especially at high capsid concentrations.

### D. Assay Testing and Verification with AAV Particles (Part 1)

This subsection describes exemplary testing data obtained with the assay protocol of Subsections B and G, the equations of Subsection C1, and samples containing commercially-available empty and full AAV2 viral particles; see FIGS. 8-11.

Two AAV standard samples were obtained from Vigene Biosciences. The “Full GFP-AAV2” sample contained AAV2 viral particles holding a genome 238 having a GFP transgene 276 under control of a CMV promoter (see FIG. 8). The “Empty AAV2” sample contained empty AAV2 viral particles.

Table 1 lists capsid occupancy data (percent full) reported by the vendor, as determined by three different techniques, namely, TEM (transmission electron microscopy), AUC (analytical ultracentrifugation), and qPCR/ELISA (quantitative polymerase chain reaction/enzyme-linked immunosorbent assay). The three techniques give substantially different values for capsid occupancy.

TABLE 1

% Capsid Occupancy (Vigene Data)

| Sample | By TEM | By AUC | By qPCR/ELISA |
|---|---|---|---|
| Full GFP-AAV2 | 71% | 93% | 51% |
| Empty AAV2 | 0.5% | 5% | Not reported |

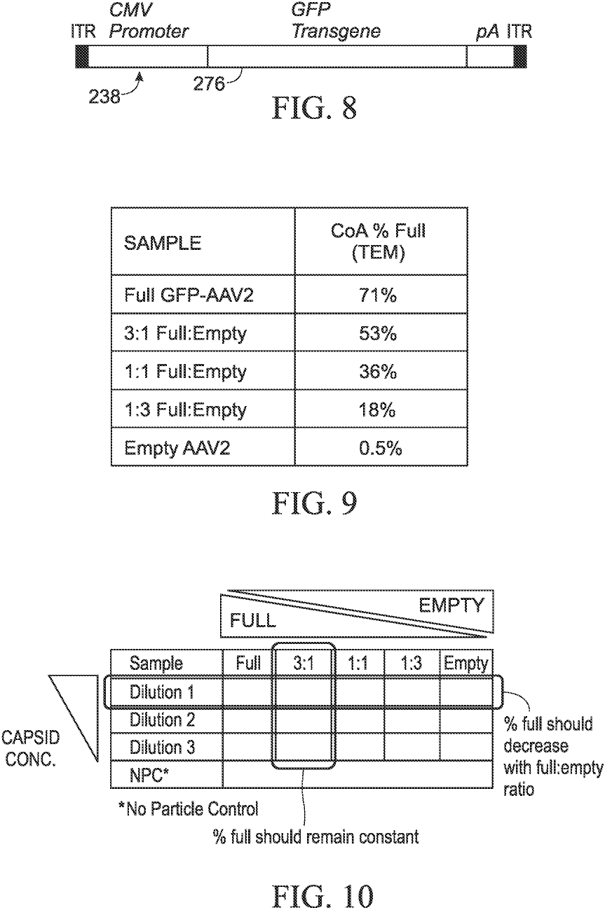

The full and empty AAV2 samples were mixed with one another at three different ratios, to provide a total of five samples for testing (see FIG. 9). The TEM-measured capsid occupancy of each of the samples, as listed on, or calculated from, the vendor’s Certificate of Analysis (CoA), is given in FIG. 9. No Particle Control (NPC) samples also were tested using the same protocol in order to subtract an NPC background signal from the samples containing viral particles (also see Subsection C1).

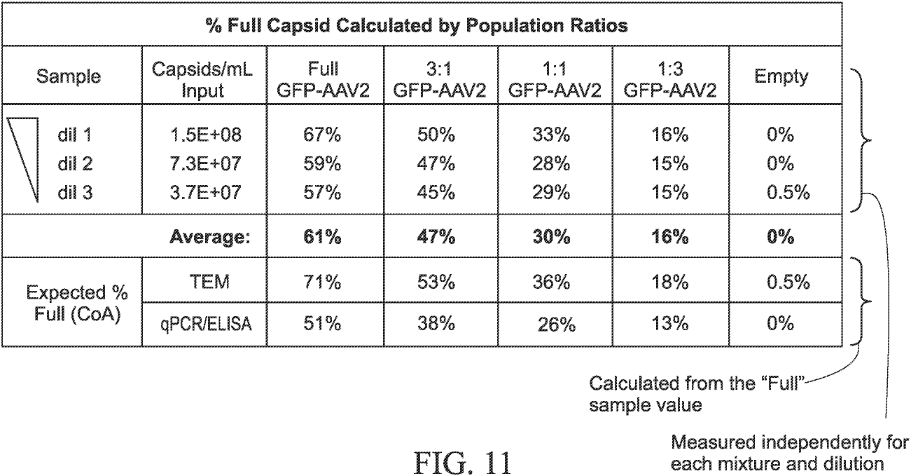

Each of the samples was tested according to Subsection B at a series of dilutions (see FIGS. 10 and 11). The partitions formed were droplets. Target amplification was performed by PCR in the droplets. Primers to the GFP transgene were used for quantification of genome copies (GC) in the droplets. The capsid occupancy values measured according to the protocol of Subsection B are consistent across a range of sample dilutions and in good agreement with the values determined by TEM.

### E. Exemplary Capsid Occupancy Calculation

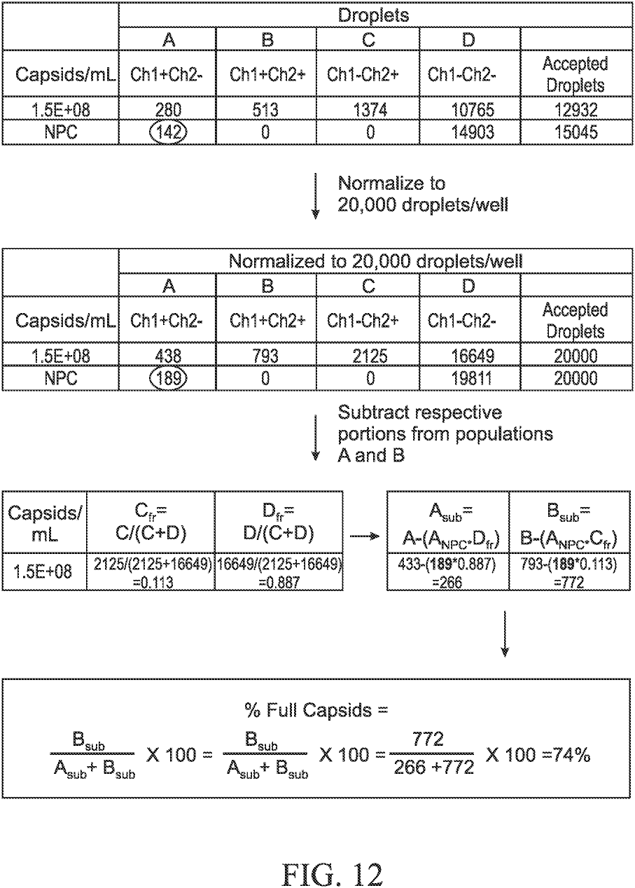

This subsection describes an exemplary calculation for capsid occupancy using equations of Subsection D and values obtained with droplets as partitions in the protocol of Subsections B and G; see FIG. 12.

### F. Assay Testing and Verification with AAV Particles (Part 2)

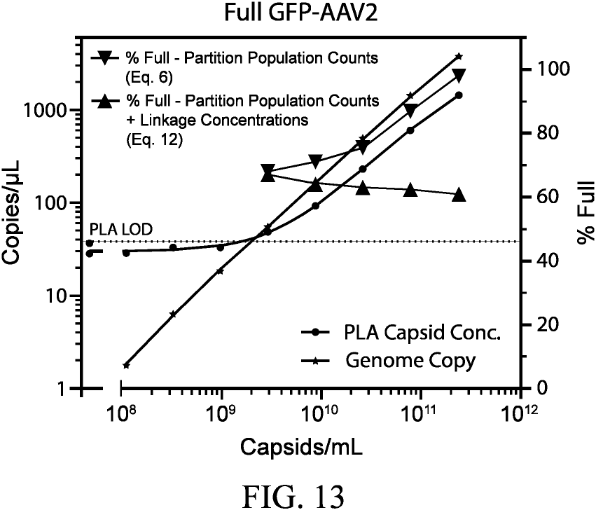

This subsection describes and compares exemplary testing data obtained with the assay protocol of Subsections B and G, the equations of Subsections C1 and C2 (with particular emphasis on Equations 6 and 12), and samples containing commercially-available AAV2 viral particles having various occupancies; see FIGS. 13-15.

FIG. 13 is a plot comparing different methods of calculating the percentage of capsids containing genomes (i.e., the % Full Capsids). Specifically, the plot shows values of the % Full Capsids calculated using Equation 6 (base-up triangles; the “partition population counts” method) and Equation 12 (apex-up triangles; the “partition population counts and linkage concentrations” method). The plot also shows concentrations of PLA Capsids (circles) and Genome Copies (stars) for comparison. The % Full Capsid is independent of the capsid’s concentration. Thus, the calculated values of % Full Capsid should remain the same at different measured capsid concentrations. In other words, the plots of % Full Capsids should be horizontal. The values of % Full Capsids calculated using Equation 6 are, in fact, sensitive to capsid concentration. In particular, the values of % Full Capsids increase with capsid concentration at higher capsid concentrations, essentially tracking capsid concentrations. This probably reflects the random co-partitioning of the capsids and genome content. In contrast, the values of % Full Capsids calculated using Equation 12 are relatively constant and consistent over a wide range of capsid concentrations. Moreover, the calculated values of about 60% Full Capsids agree with the value of 70% measured with transmission electron microscopy (TEM).

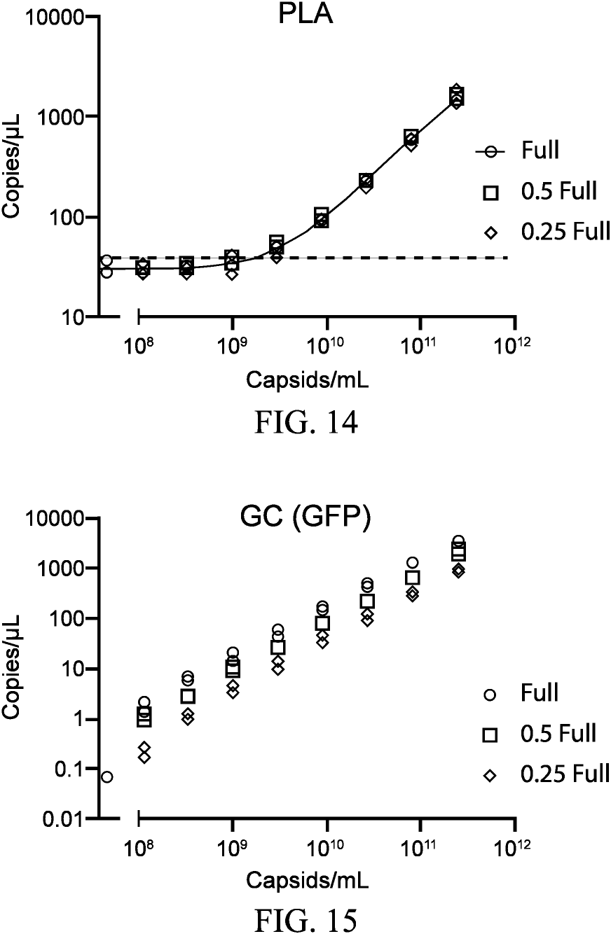

FIGS. 14 and 15 are plots obtained by performing assays over a wide range of capsid concentrations to assess suitable ranges for PLA capsid quantification, ddPCR GC quantification, and % Full Capsid calculations. All samples were tested in duplicates. The PLA standard curve was generated using both duplicates of the “Full” samples. Significantly, the recovery for most samples is within an acceptable range of 70-130%. PLA and GC capsid concentration ranges are not entirely overlapping and depend on the GC content of the sample. In general, the GC can be detected and quantified over a wider range of capsid concentrations. Here, the PLA capsid range is 2.9 × 10⁹ to >2.4 × 10¹¹ capsids/mL (FIG. 13), and the GC ddPCR capsid range is 1.1 × 10⁸ to 2.4 × 10 ¹1 capsids/mL (FIG. 14).

### G. Exemplary Reagents and Workflow

This subsection describes exemplary reagents and an exemplary workflow for an assay protocol based generally on Subsections A and B. The protocol utilizes droplets as partitions in which PCR reactions are performed for detecting capsids and a genome of AAV particles. However, the protocol may be followed for any suitable type of partition, viral particles, and amplification method.

The present disclosure suggests a workflow that allows the quantification of % Full AAV capsids using a single assay. This assay is droplet digital PCR (ddPCR)-based and does not require a calibration curve for the calculation of % Full capsids. The workflow may be modified for other viral vectors.

In the assay protocol, AAV genome copies are detected by a ddPCR reaction and measured using one detection channel of the ddPCR system. In parallel, AAV capsids are detected using antibody-mediated Proximity Ligation Assay (PLA) and detected in a second detection channel. Such a measurement results in four distinct droplet populations. The % Full capsids is calculated by a ratio between these populations.

The following steps may be completed. 1) The AAV sample is treated with DNase to digest any unencapsidated DNA. 2) The sample is diluted to reach ddPCR dynamic range. In addition to the AAV sample(s), each experiment run also includes one or more control samples that do not contain AAV particles, thereby providing a No Particle Control (NPC). The signal of the NPC is used for background subtraction. 3) A proximity ligation assay (PLA) reaction is performed. The AAV sample is incubated with two PLA probes. The PLA reaction starts with a binding interaction between the AAV particles of the samples and the two PLA probes. Each probe consists of an antibody specific to intact AAV particles, and each antibody is conjugated to an oligonucleotide. A ligation reaction of the PLA reaction then may be performed. When both PLA probes are bound to a capsid and are in proximity to each other, the oligonucleotide part of the PLA probes can form a new DNA template through a ligation reaction mediated by a short connector sequence. 4) A ddPCR reaction is prepared. The ligated samples (viral and control) are combined with a ddPCR supermix, hydrolysis probes, and primers. 5) The samples are partitioned into droplets. 6) The samples are thermocycled. Capsid lysis occurs during the first step of thermocycling, at 95° C., so that both the ligation template and the viral genome are accessible for amplification. 7) Droplets are read. The two amplified targets, one from the PLA template and the other from the viral genome, are measured by two different detection channels.

The workflow described results in droplets of four different populations (A-D). Droplets containing empty viral capsids will be PLA positive, while droplets containing full capsids will be positive for both PLA and a genome copy (GC) (also see FIG. 7).

### H. Capsid Tagging Using a Preformed Full-Length Template

This subsection describes exemplary assay reagents for, and exemplary assay configurations produced by performance of, an illustrative method according to Subsection A, where the method utilizes (i) a tagging probe including a preformed, full-length template to provide a first target for amplification, and (ii) beads as a solid support to enable one or more washing steps to remove unbound material; see FIG. 16.

A flow diagram 330 illustrates various assay configurations. A sample 332 including viral particles 334 is selected. Viral particles 334 include capsids 336 enclosing copies of a genome 338.

Sample 332 is combined with beads 331, indicated at 333. Beads 331 have a bead body 335, which may be magnetic, and a specific binding agent 337 attached to one another. Specific binding agent 337 is configured to specifically bind to a structure of viral particles 334, such as capsids 336 thereof. Any suitable number of copies of specific binding agent 337 may be attached to each bead body 335. However, the number of beads 331 may exceed the number of viral particles 334, such that a majority of beads 331 are bound to no viral particle or a single viral particle 334, as shown in mixture 339. After binding of viral particles 334 to beads 331, the beads may be washed to remove unbound material.

Beads 331 and viral particles 334 are combined with a tagging probe 344, indicated at 348. The tagging probe includes a specific binding agent 350 and a tag 362. Specific binding agent 350 allows tagging probe 344 to bind directly or indirectly to capsids 336 of viral particles 334. Tag 362 is attached, covalently or non-covalently, to specific binding agent 350 and provides a template 364. After binding and washing, indicated at 341, viral particles 334 are tagged and bead-bound, indicated at 343. Viral particles 334, beads 331, and tagging probe 344 may be combined with one another in any suitable order.

The tagged, bead-bound viral particles 334 are combined with amplification reagents, indicated at 367, and partitions 378 are formed, indicated at 379. Partitions 378 may receive an average of less than one bead 331 per partition, to reduce interference from partitions 378 receiving more than one viral particle 334. After formation of partitions 378, capsids 336 are lysed and amplification is performed, indicated at 382. A first target is amplified from template 364 and at least one other target is amplified from copies of genome 338. Amplification data is collected from partitions 378, indicated at 387. The amplification data may be visualized in a scatter plot 388. Partition populations 389a-389d (A-D) can be assigned according to target content (also see FIG. 7 of Subsection B).

### I. Illustrative Capsid Labeling

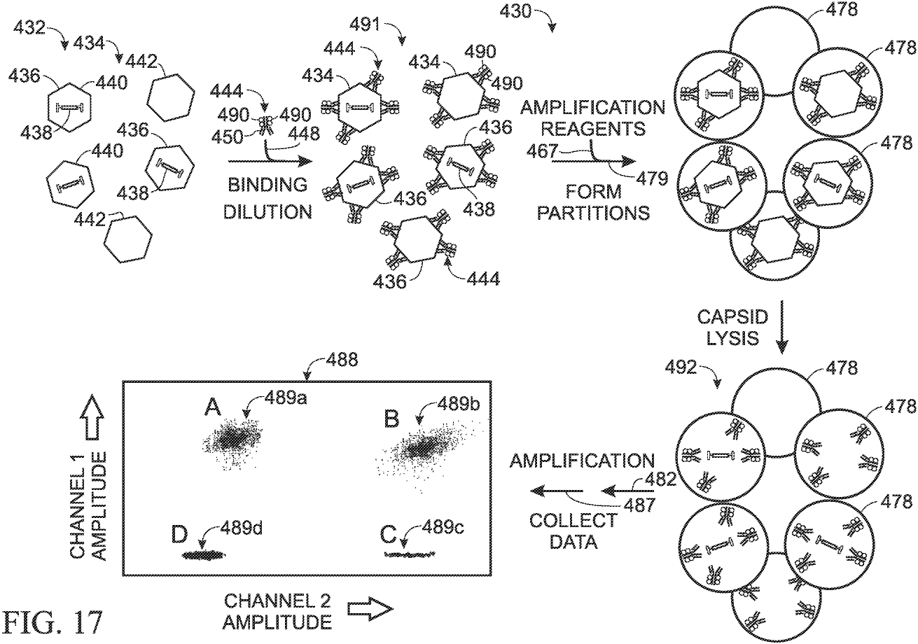

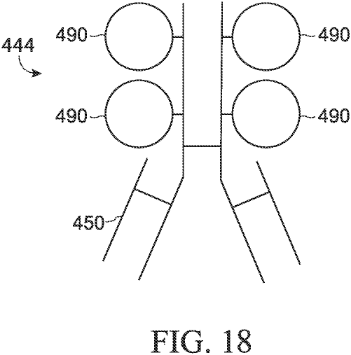

This subsection describes exemplary assay reagents for, and exemplary assay configurations produced by performance of, an illustrative method according to Subsection A, where the method utilizes a tagging probe including an optically detectable label to render capsids detectable; see FIGS. 17 and 18.

A flow diagram 430 illustrates various assay configurations of the method (see FIG. 17). A sample 432 including viral particles 434 is selected. Viral particles 434 include capsids 436 enclosing copies of a genome 438. Capsids 436 include full capsids 440 and empty capsids 442.

Viral particles 434 are combined with a tagging probe 444, indicated at 448. Tagging probe 444 includes a specific binding agent 450 connected to one or more copies of a label 490. Specific binding agent 450 directly or indirectly binds to capsids 436, which labels the capsids with label 490, as shown in configuration 491. Each capsid 436 may have multiple recognition sites for specific binding agent 450, such that multiple copies of tagging probe 444 can bind to the same capsid 436, to increase the signal detectable from label 490 for a given labeled viral particle 434.

Viral particles 434 are combined with amplification reagents, indicated at 467, and partitions 478 are formed, indicated at 479. The amplification reagents are sufficient for amplification of one or more targets from genome 438.

Capsids 436 are lysed to produce configuration 492. Amplification is then performed, indicated at 482, and tag-related data and amplification data are collected from partitions 478, indicated at 487. The tag-related data (detected from label 490 in channel 1) and the amplification data (detected from at least one amplification probe for at least one genome target in channel 2) may be visualized in a scatter plot 488. Partition populations 489a-489d (A-D) can be assigned according to tag/target content (also see FIG. 7 of Subsection B).

### J. Compositions and Methods for Detecting Viral Components

This subsection describes further aspects of compositions and methods for analyzing viral particles. The ability to study, test for, quantify, and treat viruses is of primary concern for human health, and methods and/or technology to quantify said viruses aid in this endeavor. There is a need for improved compositions and methods for detecting and quantifying viral components and functional viruses.

Provided herein are compositions and methods for analysis of viral samples. In certain embodiments, provided are compositions and subsequent use of reagents to quantify viral titer and, in some cases, functional titer, in unknown samples. In certain embodiments, provided herein are compositions and uses of the compositions for the simultaneous detection of viral DNA and viral capsid allowing for the extrapolation of, e.g., viral integrity, infectivity, and infective titer. The compositions and methods may be used with appropriate instrumentation to aid in analysis. In some embodiments, the instrumentation is digital analysis instrumentation.

Viruses are infectious agents that require a living cell for replication. Generally, they comprise of a protein shell (or capsid) and a nucleic acid genome. The genome may comprise either single- or double-stranded DNA or RNA. Furthermore, depending on the class, viruses can be enveloped or non-enveloped. The nucleic acid genome of viruses is extremely diverse. As a group, viruses contain more structural genomic diversity than any of the other kingdoms of life.

Capsids are the protein shell of a virus enclosing its genetic material. There are a number of capsid-types that may be generally classified by their structure (either icosahedral, prolate, or helical) and then further classified by the underlying protein sequence of the capsid components.

Enveloped viruses have a phospholipid bilayer coating their protein shell, the result of budding at a cell’s plasma membrane following viral replication. All viral surfaces, regardless of envelope status, display specific proteins that facilitate host cell adsorption and internalization.

Single-stranded viral genomes, in which the complementary strand is missing may be of either sense (positive or negative) depending on the viral class and their downstream mechanism of replication once their host has been infected.

Furthermore, viral genomes may exist either as linear or circularized in the macroscopic structure.

Additionally, viral genomes range in size and as a result in the number of proteins that they encode. One of the smallest known viruses comprises a two kB genome that encodes only two proteins, while one of the largest known viruses has a genome of 2 MB encoding about 2,500 proteins.

Furthermore, RNA viruses typically demonstrate higher mutation and evolution rates as they propagate. This is due to the inherent lack of fidelity that RNA-dependent polymerases demonstrate over typical DNA-dependent polymerases. As a result, these viruses also tend to have smaller genomes due to a strong balancing selection between beneficial mutations and accumulation of detrimental mutations during replication.

As a result of these different properties and/or characteristics of viruses, the methodologies, reagents, and technologies utilized for their analysis can be targeted.

Naturally-occurring viruses often lead to a disease state in the host in which they are replicating. Such examples for humans include the common cold, influenza, chickenpox, HIV, rabies, Ebola, and the like. The ability of the viruses to cause disease is termed virulence. Viruses have different mechanisms by which they produce disease in their host organism which may relate to the mechanism of viral replication. Lytic replication, in which the cells explode (or lyse) once the virus has replicated to a sufficient state and triggered release, leads to immediate death for single cellular organisms or a disease state once a sufficient number of cells in a multicellular organism has been compromised.

Some viruses may exist in a dormant or latent state in the host where, while latent, they exist relatively harmlessly within the organism. Other times, viruses may cause chronic infections where the host is unable to remove the virus.

Additionally, due to the innate ability of viruses to deliver genetic material to the cell, researchers have utilized viruses as delivery vectors in the hopes of developing targeted gene therapies.

Viral particles can be engineered for the delivery of genetic material to the cellular cytoplasm and nuclei, with engineered vehicles described as viral vectors. Therapies that alter a patient’s genome in and ex vivo through the replacement or repair of mutated genes or redirection of the body’s response toward diseased cells are emerging as the next generation of therapeutics. Unfortunately, there is a lack of consensus around viral vector characterization assays critical to the safety and efficacy of such therapies. Accurate quantification of viral titer, or the determination of virus count in a given volume, is required for patient dosing. Interrogation of active fraction, or the proportion of viral genome-containing capsids, provides insight into the risk of immunogenicity and variability in gene transfer efficiency. Imperfect sample processes can result in empty or partial capsids, where only a portion of the viral genome (vg) is incorporated. Empty and partial capsids are incapable of infection and, therefore, successful gene transfer, limiting the portion of sample that is “active.” For this reason, empty and partial capsids are considered a sample impurity and must be closely monitored from batch-to-batch.

A singular workflow with the ability to simultaneously assess viral titer and active fraction could positively influence vector and plasmid design, manufacturing process development and scale-up, and clinical batch characterization. Further, such an assay could have a beneficial impact on a wide range of efforts in both academic and industrial research and development, medicine, public health, and manufacturing, including vaccines, antiviral therapies, recombinant protein production using viral vectors, viral load management, and the monitoring of viral outbreaks.

Viral titer can be described as physical or functional. Physical titer typically involves quantification of viral nucleic acid, protein, or physical particles. Functional titer, also known as infectious titer, is the measurement of viral particles that can successfully infect cells. Infectious titer will often be lower than physical titer, as not all detectable viral components in a sample are properly assembled into an infectious unit, and is considered the gold standard in patient dosing.

Common examples of functional titer assays include plaque assays and 50% tissue culture infectious dose (TCID₅₀) assays. Plaque assays involve infecting a monolayer of cells with a viral dilution, which is then sealed with a top layer of agar to prevent viral spread beyond neighboring cells, and counting the number of visible viral infections, or plaques, following a period of incubation. TCID₅₀ assays also treat cells with a range of viral concentrations, but in a microplate. Visual inspection then determines the virus concentration at which 50% of wells in the microplate show evidence of cytopathic effect, regardless of the quantity of events within the well. While functional titer assays are highly sensitive, yield absolute measurements of infectious units, and do not require specific reagents, they are labor intensive, require extended protocols (typically 5+ days) and can be user-subjective.

Physical titer can be quantified using methodologies such as quantitative polymerase chain reaction (qPCR) and enzyme-linked immunosorbent assays (ELISAs). qPCR is an analog assay in which the concentration of target nucleic acid in a bulk sample is determined relative to a standard curve prepared using reference material. Quantification in relation to a reference increases the potential for variability across assays, users and labs, and limits assays to well-characterized standards. Further, poor amplification efficiency of either the target or reference nucleic acid can result in the over- or under-estimation of the target concentration. ELISAs measure viral proteins through specific antigen capture. First, the antigen is directly or indirectly (i.e., bound by a capture antibody) immobilized on a microplate, then the antigen is bound by a detection antibody that facilitates an enzymatic or fluorescent readout. These methods enable more rapid results with decreased labor while maintaining acceptable sensitivity levels. However, the benefits come at the cost of relative, not absolute, quantification, a need for specific reagents and a lack of insight into infectious units.

Physical titer may also be determined by directly counting virus particles using techniques such as flow cytometry (FCM), transmission electron microscopy (TEM) and liquid chromatography (LC). FCM is able to rapidly provide absolute quantification at a low labor cost and, depending on the protocol, can quantify infectious units, however this comes at reduced sensitivity compared to the methods previously discussed. The most common FCM protocols involve staining the vg with a fluorescent intercalating dye, or the detection of virus-infected cells via reporter genes or viral proteins. TEM stains viral capsids to enable direct counting, the study of viral morphology, and designation of empty versus full capsids. LC requires that a sample is prepared in a solvent, or liquid phase, and then pumped into a stationary phase made up of adsorbent material. The sample’s interactions with the liquid and stationary phases affect the time that it takes to elute, or its retention time, which can be used to identify analytes in a heterogeneous mixture. LC is seen as advantageous in the manufacturing process as results are rapid and can be generated using samples from any stage of the production process (i.e., crude samples). Methods for interrogating viral particles through their physical properties, generally, are lower throughput and have more specific use cases.

Several methods previously discussed for viral titer quantification can be adapted for the assessment of active fraction, such as qPCR, ELISA, TEM, and LC. Active fraction is the percentage of virus in a sample capable of introducing its genetic material into target cells, often interpreted to be the proportion of capsids containing viral genome. qPCR and ELISA can be used in conjunction to estimate the ratio of vg to viral capsids as a readout of active fraction; however, while this approach can be easily incorporated into existing workflows, it lacks accuracy and precision. TEM enables visual determination of empty and full particles. While data acquisition for TEM is relatively rapid, data interpretation is often highly time-consuming and requires a skilled technician. High-performance liquid chromatography (HPLC), which relies on a pump rather than gravity to generate the pressure required to move a sample through the phase column, separates particles by size. A method not previously discussed, analytical ultracentrifugation (AUC), relies on density-based sedimentation to distinguish between subpopulations in a heterogeneous sample. AUC provides high resolution and negates the need for reference material, however it does require large sample volume, relative to the production scale that many researchers are working at.

Digital assays have become an important tool for quantifying chemical or biochemical species in scientific research and clinical diagnostics. In a digital assay, analytical targets within a sample are partitioned into a plurality of subsamples according to a known probability distribution, in many cases a Poisson distribution. A method is applied to determine whether each of the subsamples contains the analytical target(s) of interest. Measuring both the total number of subsamples as well as those which contain or those that do not contain any target, and comparing to the probability distribution allows for a user to infer the quantity of each analytical target in the original sample. The methods are termed “digital” because only a discrete measurement is taken on each subsample, i.e., whether that subsample contains each analytical target or not.

Digital assays have a number of key advantages over traditional means of quantifying the number or concentration of an analytical target. First, because the method directly counts the number of subsamples occupied by, or unoccupied by, an analytical target and compares that to a probability distribution, a direct measurement of the original quantity of an analytical target can be made without reference to a calibration standard. This is especially valuable when construction of such a standard is difficult to do repeatedly, accurately, or at all (no available reference material). Second, because only the presence of an analytical target in each subsample must be measured, small or moderate variation or noise in the detection signal does not alter the final determination of presence (and thus quantity/concentration) of the target in the sample. Third, because the volume of each subsample is inherently smaller than the original sample, the relative concentration of the analytical targets in each subsample that contains the targets is higher than in the bulk sample. In cases with many subsamples and low quantities of analytical targets, the increase in concentration can be multiple orders of magnitude, increasing concentration-dependent sensitivity.

In digital PCR (dPCR), the analytical target(s) of interest is a sequence of DNA or cDNA, if RNA is to be quantified. Nucleic acid molecules containing the sequence of interest are distributed amongst subsamples which are then subjected to PCR thermal cycling. Each subsample is optically interrogated to determine whether the sequence of interest amplified, and the original quantity of DNA molecules with the sequence(s) of interest can be inferred from the probability of Poisson distribution. Note that quantification is a discrete measurement dependent on end-point amplification and, therefore, less susceptible to amplification inhibitors than qPCR. Further, quantification is independent of reference standards, reducing interassay variability compared to qPCR.

PCR, with the proper assay design and optimization, is able to provide quantification of viral physical titer and, in some cases, even infectious units. However, researchers seeking more detailed information, such as active fraction, would find useful a single assay that is able to quantify both vg and capsid concentrations. While qPCR may offer solutions for parallel sample processing of either vg or capsid concentrations, dPCR allows for concurrent genome and capsid interrogation of partitioned sample, enabling absolute quantification of viral titer and active fraction in a single assay.

Parallel sample processing quantifies capsid concentration in an unprocessed sample, taking advantage of the capsids’ external physical properties, and vg concentration in a second, processed sample where the capsids have been disassembled to release the vg. Capsids can be disassembled using a variety of techniques including, but not limited to, heat treatment, enzymatic digestion (i.e., proteinase K, trypsin, cathepsin B), sonication, surfactants, acids and bases (i.e., Tween20, guanidine hydrochloride). Examples of PCR vg quantification methods include, but are not limited to, intercalating dyes, hydrolysis probes and molecular beacons. These methods could be used in parallel with PCR-based capsid quantification techniques, such as quantification of oligonucleotide-binding molecule conjugates, and PCR-compatible techniques, such as direct and indirect fluorophore-labeled binding molecules.

Concurrent sample processing allows for capsid and vg quantification in the same sample, seeking to identify subsamples that are positive for both capsid and vg to confirm proper assembly/loading and, subsequently, active fraction. Most of the vg quantification techniques identified for qPCR can be transitioned, with some optimization, to concurrent sample processing in a dPCR format. dPCR concurrent sample processing generally adopts the following workflow: labeling of capsids in bulk sample, partitioning sample into a sufficiently large number of subsamples, disassembling capsids while retaining the ability to detect capsid label and, finally, detecting vg and capsid within subsample. Additional insight into manufacturing process efficiencies is provided in the form of vg only or capsid only subsample readouts. A further level of precision may be introduced by using multiple sets of PCR primers for the viral genome, each of which is specific to a different part of the genome in the same partition; if the sets are differentially labeled, it is possible to estimate the number of complete copies of the viral genome by counting partitions containing detectable label from all primer sets, and the more primer sets that are included, the more accurate the assessment of completeness (and, presumably, infectivity) can be. In some cases where the viral genome is relatively small, e.g., AAV, two sets of primers, e.g., for distal parts of the genome, may be sufficient. In other cases, more sets of primers, e.g., 3, 4, 5, 6, 7, 8, 9, 10, or more than 10, may be sufficient. In some cases, e.g., where a process is known to produce mostly or entirely intact copies of a viral genome, a single set of primers can be sufficient.

In general, compositions and methods provided herein are directed at analyzing partitions (that is, a relatively small portion of a parent sample) that may contain viral nucleic acid; capsid; both viral nucleic acid and capsid; or neither viral nucleic acid nor capsid. The analysis will detect which class the partition belongs to. In certain embodiments, methods and compositions for preparing partitions are also provided.

If a sample comprising viral particles that include both capsid and viral nucleic acid, and potentially also comprising capsid without viral nucleic acid and/or viral nucleic acid without capsid, is subdivided into partitions, single partitions may include no viral components; capsid only; viral nucleic acid only; viral capsid and viral nucleic acid from a single viral particle; and viral capsid and/or nucleic acid from multiple viral particles. The latter will be uncommon if a sufficient number of partitions is produced from the sample. In one analysis, if a sample is subdivided into a number of partitions and each of the partitions is classified as containing no viral components; capsid only; viral nucleic acid only; or viral capsid and viral nucleic acid (which can be from single or multiple viral particles but which, if the sample is subdivided into a sufficient number of samples, will be almost entirely from single viral particles), then the portions of the sample that comprises a full capsid, i.e., capsid and viral nucleic acid, or an empty capsid (capsid only) can be determined. Initial concentrations of each in the original sample can also be determined based on the number of partitions of each class and, in some cases, the volumes of the partitions. In another analysis, e.g., where a plurality of sets of primer pairs are used, portions of the sample that contain a partial capsid, i.e., capsid and incomplete viral nucleic acid; full capsid, i.e., capsid and complete viral nucleic acid; and empty capsid (capsid only) can be determined. Initial concentrations of each in the original sample can also be determined based on the number of partitions of each class and, in some cases, the volumes of the partitions. In some cases, full viral particles may be considered infective and the active fraction determined.

### 1. Capsid Detection and Quantification

Typically, viral capsid (protein) or a component associated with the capsid (lipid bilayer) is tagged with a component sufficiently specific for the particular assay being used. The tagging component binds to viral capsid or lipid bilayer and is directly or indirectly detectable. Directly detectable tagging components can be labeled with a label that is detectable either on its own or after certain modification (see, e.g., FRET, described elsewhere herein). The label may be an intrinsic part of the tagging component or may be bound to the tagging component; binding of the label may occur before binding of the tagging component to the capsid or lipid bilayer (e.g., fluorescently labeled antibodies) or after (e.g., a labeled antibody specific to the tagging component, or fluorescent label associated with PCR of a tagging oligonucleotide). In addition, the binding of the tagging component to capsid or lipid bilayer may be covalent or non-covalent.

In certain embodiments, viral capsid is tagged in a bulk sample, i.e., prior to formation of partitions. The sample is then divided into a plurality of partitions, some of which will contain capsids. The capsid is then treated, as described elsewhere herein, to sufficiently disrupt capsid integrity such that nucleic acid present within the capsid, if any, is exposed sufficiently for amplification (if used) and detection reactions to take place within individual partitions. Before formation of partitions, reagents sufficient for the desired reactions are present, so that partitions individually contain the reagents. Alternatively, partitions containing reagents or other appropriate components may be fused with sample partitions prior to reactions or other manipulations. In the case of capsid tagged with a component that comprises an oligonucleotide to be amplified, partitions will contain the appropriate sets of primers and PCR reagents, e.g., a master mix. As described elsewhere, one set of primers may be used specific for viral nucleic acid, or more than one set of primers may be used, each specific for a different sequence of viral nucleic acid. In addition, in embodiments in which capsid is tagged with a component comprising an oligonucleotide to be amplified, one or more sets of primers specific to the capsid tagging oligonucleotide will be present in the partitions. Thus, provided herein in some embodiments are compositions comprising a subsample, e.g., partition, of a larger bulk sample containing viral particles, wherein the subsample, e.g., partition, comprises a tagged capsid component of the virus, e.g., a tagged capsid protein or lipid bilayer, as described herein, and reagents sufficient for performing polymerase chain reaction on viral nucleic acid, if present. In certain embodiments, the subsample will also comprise viral nucleic acid. The reagents sufficient for performing PCR on viral nucleic acid will include at least one set of primers specific for a sequence of the viral nucleic acid; in certain embodiments, the reagents sufficient for performing PCR on viral nucleic acid will include at least 2, 3, 4, 5, 6, 7, 8, 9, or 10 different sets of primers, each specific for a different sequence of the viral nucleic acid. In certain embodiments in which a capsid tag comprises an oligonucleotide to be amplified, the subsample also contains at least one set of primers specific to this oligonucleotide. Appropriate labeling components, depending on the specific assay, may also be present. In certain embodiments, at least 10, 50, 100, 200, 500, 1000, 2000, 5000, 10,000, 20,000, or 50,000 subsamples, e.g., partitions, of the original sample are present in the composition. The volume of the subsamples may be any suitable volume, depending on the further treatment of the subsamples, e.g., 0.01-100 nL, or 0.1-10 nL, or 0.1-5 nL, or 0.1-1 nL. In certain embodiments, the composition comprises subsamples containing tagged capsid, as described above, as well as subsamples that do not contain tagged capsid. In certain embodiments, the virus is an AAV.

Capsid detection and/or quantification may be based on specific or non-specific interactions with viral capsid, e.g., viral protein, or viral lipid bilayer. Specific interactions involve binding to specific moieties of the viral capsid; binding to non-viral capsid moieties does not occur to a significant degree, e.g., a degree that would interfere with the desired analysis; specificity may be specific to viruses, specific to a class of viruses (e.g., AAV), or specific to one or more subclasses of viruses (e.g., AAV serotypes). Non-specific interactions involve binding to moieties of the viral capsid that may also be present in significant quantities in non-viral substances, e.g., non-specific protein binding or lipid bilayer insertion. In samples that are relatively pure, e.g., samples of transgenic AAV or other types of viral particles that are artificially produced, the majority or totality of protein and/or lipid present may be that of capsid and/or lipid bilayer, thus non-specific interactions are sufficient to detect and quantify capsid. In samples that may contain other proteins and/or lipids than those from viral particles, specific interactions are preferable, i.e., interactions that involve specific binding to viral capsid component or specific binding to specific viral serotypes.

Capsid detection and/or quantification can take advantage of physical surface properties such as exposed amino acids, antigens or, in the case of enveloped viruses, the lipid bilayer. In certain cases, binding to capsid may be non-specific. For example, compositions and methods that detect lipid bilayers, discussed in more detail below, will generally detect a lipid bilayer encapsulating a viral particle and, potentially, other lipid bilayers that may be present. Detection molecules, such as fluorescent dyes or oligonucleotides, can be conjugated directly to the capsid’s surface at the site of exposed amino acids. Conjugation can be performed, for example, using well-studied click chemistry reactions like alkyne-azide cycloaddition (i.e., DBCO-azide).

If more specific methods are necessary, one or more surface capsid antigens can serve as a binding moiety to enable capsid quantification to determine total capsid count; these can be serotype specific or ambiguous, recognizing partial or intact capsids. Potential binding molecules include, but are not limited to, antibodies, peptides, single-chain variable fragments (scFvs), nanobodies, aptamers, lipids and nucleic acids. Selection of appropriate binding molecules will likely be dependent on virus type, serotype, and assay commercialization requirements. The binding molecules highlighted below focus on AAV-specific molecules, given the interest in developing a singular assay for vector characterization to be used in the gene and cell therapy (GCT) space.

### 2. Examples of AAV Binding Molecules

AAV Receptor (AAVR): Type-I transmembrane protein KIAA0319L, also known as AAVR, has been identified as a direct binder of AAV serotype 2 (AAV2), crucial to disease susceptibility. Genetic ablation of AAVR protects cells against AAV2 infection. Further, additional studies revealed AAVR to be a critical host factor for AAV serotypes 1, 3B, 5, 6, 8 and 9, making it an ideal binding molecule for non-specific detection of most AAV capsid serotypes, i.e., specific for AAV but non-specific for AAV serotype. AAVR contains 5 polycystic kidney disease (PKD) domains which mediate cell binding, however only PKD 1-2 appears to be required for infection. The use of a shortened construct of PKD 1-2 AAVR, which occupy positions 309-400 and 408-497, respectively, could be advantageous with regards to binding capacity and supply chain logistics. The shortened construct could prove to be further advantageous given its increased binding capacity over full-length AAVR; studies have shown that binding capacity is increased at least 14-fold, dependent on model adjustments, by cryo-EM. Thus, compositions and methods provided herein can, in certain embodiments, include an AAVR, or a fragment thereof, such as a shortened construct of PKD 1-2 AAVR, so long as the AAVR or fragment binds to AAV capsid with sufficient specificity and tightness to be used in the methods provided herein. Such a moiety can be used to tag a capsid with desired specificity. In certain embodiments, such a moiety may be linked to a detectable label. In certain embodiments, the moiety may be linked to an oligonucleotide capable, by itself or in combination with other components, of being amplified by PCR.

A20 monoclonal antibody (mAb): A20 mAb is the most widely studied mAb against AAV2. A20 mAb is preferred for its specificity towards only intact, assembled capsids given that it recognizes a conformational epitope defined by protein tertiary structure. It is a neutralizing mAb, meaning that upon binding it renders infectious particles ineffective. While A20 mAb is also known to bind AAV3B in addition to AAV2, it does not bind other serotypes and, therefore, can be useful in characterizing a heterogeneous sample. Compositions and methods provided herein can, in certain embodiments, include an A20 mAb, or a fragment or aptamer thereof, or a combination of these, so long as the antibody, fragment, or aptamer, binds to AAV capsid with sufficient specificity and tightness to be used in the methods provided herein. Such a moiety can be used to tag a capsid with desired specificity. In certain embodiments, such a moiety may be linked to a detectable label. In certain embodiments, the moiety may be linked to an oligonucleotide capable, by itself or in combination with other components, of being amplified by PCR.

PCR-compatible fluorescent readouts can be used to non-specifically or specifically label capsid proteins. Non-specific protein labeling, such as with Sypro® Orange (Life Technologies), may be employed for capsid labeling so long as sample protein impurities are minimal. Specific fluorescent protein labeling can be achieved through the association of a fluorophore with a surface binding molecule, either directly or indirectly. Directly-labeled conjugates can be prepared using random or site-specific conjugation strategies. Indirect labeling methods include a fluorescently-tagged secondary binding molecule, specific for the primary binding molecule, or a proximity readout. Sufficient binding with primary or secondary fluorophore conjugates may be followed by thorough washing to remove unbound fluorophore in order to prevent a false positive result. Alternatively, fluorescent readouts that depend on proximity, such as Forster resonance energy transfer (FRET) or PerkinElmer’s AlphaScreen™ can be used without washing. In proximity readouts, a pair of indirect labeling molecules must be appropriately close in distance to produce a positive signal; the likelihood of a positive signal from free, unbound pairs in the reaction solution is low. Following binding, and any necessary wash steps, the sample can then be partitioned into subsamples and intact capsids quantified.

In situations where a positive readout is dependent on successful binding of different targets, a multiplex approach should be taken. For example, molecules can be directly labeled with different fluorophores, each corresponding to a particular surface target, or unique FRET pairs. The choice of exact fluorescent panel will vary based on potential fluorometer limitations of the instrument and, for FRET and AlphaScreen™, the expected distance between bound molecules. In either case, a positive signal determination would be dependent upon the successful binding of all targets.

Preparation of conjugates: Proteins, including antibodies, can be labeled using random or site-specific strategies. The most well-established protein labeling technique is random labeling of primary amines on lysine groups. One example is a copper-free click chemistry strategy where primary amines are labeled with DBCO and subsequently reacted with an azide to form a covalent linkage. As there can be numerous primary lysines exposed on a protein’s surface, the degree of labeling is difficult to control and the final product is a heterogeneous species. For homogeneous products, site-specific strategies are usually preferred. These typically involve chemical or enzymatic modifications to create or expose reactive amino acids. Non-exhaustive examples include the expression of a genetically modified active site (i.e., HaloTag), site-directed mutagenesis for the introduction of cysteine through reactive thiols for subsequent coupling with maleimide, and enzymatic formation of isopeptide bonds between amine and amide groups of lysine and glutamine residues, respectively. Formation of these isopeptide bonds is catalyzed by the enzyme transglutaminase and often requires the insertion or exposure of a glutamine residue.

Wash steps: Wash method selection will primarily depend on required throughput and sample volume. Potential methods include spin columns, magnetic bead capture and coated plate capture. Spin column methods enable the processing of greater sample volumes compared to plate-based methods but, for most commercial kits, this comes at the cost of decreased throughput. It should be noted that for all wash methods, if the molecules necessary for capture and detection are the same, then false positives are likely.

Spin columns commonly separate samples either by molecular weight or sample-resin interactions. Columns which use molecular weight cutoffs allow passage of all molecules below a given molecular weight while non-specifically retaining all molecules above the given cutoff. Spin columns which take advantage of specific sample-resin interactions make use of sample affinity tags. Affinity tags, such as glutathione S-transferase (GST), histidine (HIS) and biotin, are often used in sample purification during production. Tagged molecules are captured within the spin column resin while the remainder of the sample is washed away. Captured molecules are then eluted, or released, from the resin using an appropriate buffer or competitive displacement. Affinity resins include glutathione, metal ions and avidin for GST, HIS and biotin, respectively.

Magnetic bead capture can be used to separate a sample based on binding of affinity tags, similar to spin columns, or specific antigens to a coated magnetic microbead. Magnetic beads “pull down” sample bound to their surface when placed next to a strong magnet. This allows for unbound molecules to be washed away whilst retaining bound molecules. Magnetic bead capture can be performed as a tube-based or plate-based method.

Coated plate-based capture can bind and capture affinity tags and surface antigens using a coated microplate. Unbound molecules can then be washed away. Captured molecules are eluted off the plate using an appropriate buffer or competitive displacement, similar to spin columns.

While many of the capture method interactions discussed above are robust, the capsid-binding molecule interaction may be insufficient to withstand washing. Accordingly, crosslinking can be performed to ensure that capsids are retained during wash steps.

Given that the biotin-avidin interaction is the strongest known non-covalent interaction between a protein and ligand, a modified interaction can be considered to facilitate milder elution conditions (i.e., desthiobiotin or monomeric avidin).

Proximity fluorescent readouts: FRET occurs when energy transfers from a donor fluorophore to an acceptor molecule (fluorophore or quencher) with spectral overlap. As such, a FRET pair can be used as an indirect readout of capsid surface-bound molecules, so long as the targets are sufficiently close (1-10 nm).

AlphaScreen™ is an alternate homogenous proximity assay with a greater physical range than FRET, up to 200 nm, that assumes the same assay design - unbound molecules are sufficiently distant to prevent the generation of a signal. Fluorescent excitation of the AlphaScreen™ donor bead results in the emission of a singlet oxygen, which triggers a chemiluminescent reaction on the surface of a nearby acceptor bead and, ultimately, a measurable fluorescence emission. Both FRET and AlphaScreen™ technologies enable simplified, no wash assay protocols for the detection of capsid-specific surface molecules.

Oligonucleotides conjugated to surface binding molecules offer a PCR-based readout for capsid quantification through the generation or protection of intact primer binding sites. Positive readout would be determined using fluorescent primer PCR sets.

Oligonucleotide-binding molecule conjugates: Binding molecules that are labeled with oligonucleotides at the terminus opposite the target offer readouts where the primer binding site has either been generated or protected using an appropriate ligation or nuclease enzyme, respectively.

For primer site generation, two binding molecules conjugated to oligonucleotides that, together, form a primer binding site would need to bind to the capsid surface at a sufficiently close distance to facilitate the joining of nucleic acid strands by ligation. Depending on ligation conditions, it’s conceivable that a wash step could be unnecessary given the higher energy barrier for ligation of unbound conjugates in solution compared to ligation of bound conjugates. However, a wash step may be necessary if unbound nucleic acid conjugates are susceptible to ligation. If this is the case, capsids may first be treated with only one oligo conjugate variety and, subsequently, have unbound, excess conjugate washed away ahead of treatment with the second oligo conjugate variety required for ligation. Alternatively, or in addition, washing may be performed after the second oligo conjugate has been introduced.

For primer site protection, binding molecules conjugated to an oligonucleotide would serve as the target for a complementary, free oligonucleotide, forming a double-stranded product. This double-stranded product is protected against cleavage by nucleases targeting single-stranded nucleic acids, such as S1 nuclease. If the sample is treated with oligonucleotide conjugate in excess, then a wash step will be required to prevent false positive signals.

While all strategies discussed thus far are applicable to any capsid with surface antigens, there are additional quantification options for enveloped viral capsids such as herpes simplex virus and retrovirus, including lentivirus, which take advantage of their lipid bilayers. Probes that spontaneously insert into the lipid bilayer can be quantified by using readouts, depending on wash preference.

Lipid bilayer labeling methods: Probes that spontaneously insert into the lipid bilayer can be quantified using a fluorescent readout. The fluorescent readout can either be the result of a stable signal (i.e., a direct fluorophore conjugate) or dynamic signal that is altered based on dye proximity.

Direct fluorophore conjugates composed of fluorophore dyes and sterol probes, such as cholesterol or alpha-tocopherol probes, can be used to visualize an intact enveloped virus. Inserted probes will generate a signal independent of the number and proximity of those incorporated. Any free, unbound probe must be removed with sufficient washing to prevent a false positive signal.

Dyes whose fluorescent signal emission is dependent on proximity can be used without wash steps. While there are a number of methods for proximity-dependent emission, pyrene excimer formation and octadecyl rhodamine B self-quenching are two examples. At sufficient incorporation levels, pyrene-labeled fatty acids exist as an excimer. The fluorescent emission of pyrene excimers is shifted relative to that of pyrene monomers, the expected state of free probe in solution, which can be used as a positive signal for the presence of enveloped capsids. Octadecyl rhodamine B self-quenches upon dimer formation within the lipid bilayer, enabling capsid quantification through changes in the fluorescent signal of monomers versus dimers. Quantification techniques which rely on these proximity dyes may be optimized to ensure the release of the viral genome without capsid disassembly.

### 3. Viral Genome Detection and Quantification

For proper quantification, the viral genome can be released from the viral capsid. This can be achieved through a number of capsid disassembly methods, some of which have been highlighted above. If only encapsulated copies of a genome are to be quantified, samples may be treated with DNase | pre-treatment to digest any non-encapsulated nucleic acid. Given imperfections in the Gene and Cell Therapy manufacturing process, viral genome quantification may strive to confirm not only viral genome count but also genome integrity and sample purity. This can be done through specific target and signal amplification.

Fluorescent primer-probe set: Polymerase chain reaction (PCR) relies on the annealing of a single stranded oligonucleotide primer to a complementary ssDNA target sequence. Bound primers serve as a starting point for DNA polymerase, which synthesizes a complementary strand of DNA to produce double-stranded DNA (dsDNA). The target is then amplified through multiple cycles of denaturation of the dsDNA product to allow for ssDNA primer annealing and subsequent DNA polymerase elongation. RNA samples may be reverse transcribed into cDNA ahead of PCR amplification.

A dsDNA intercalating fluorescent dye, such as SYBR™ Green | (Life Technologies), can be used to generate a positive/negative signal readout using the final dsDNA product; the structure of the dye is altered upon binding to dsDNA, which results in increased fluorescence. However, this readout does not provide granular information on specific targets if multiple primer sequences are being used. Primer multiplexing could prove advantageous when examining a viral genome for genome integrity (i.e., confirming that two distal sequences of interest are present) or for sample purity (i.e., primers specific to plasmid backbone).

To better understand whether the complete genome, or any host nucleic acid, was encapsulated, fluorescent hydrolysis probes corresponding to each target may be used. In this case, an oligonucleotide probe with a 5′ fluorescent reporter and 3′ quencher molecule binds downstream of the PCR primer. Upon separation of the reporter and quencher molecules by DNA polymerase during strand elongation, the fluorescence of the reporter molecule increases. Various combinations of fluorescent reporter molecules can be used in a multiplexed readout to provide valuable feedback on manufacturing and downstream processes. If the number of desired targets surpasses the capabilities of the fluorometer, the inclusion of one or more chemiluminescent readouts, or any other suitable labeling, may be considered. By making the signal readout of a final target dependent upon a chain chemiluminescent reaction, one can assume the presence of all intermediate targets.

Molecular Beacon: The reporter-quencher principles of hydrolysis probes can be applied to an alternate probe design, the molecular beacon. Molecular beacons are oligonucleotide probes with a stem loop structure. The probe consists of a target sequence flanked by two complementary stem sequences with 3′ reporter and 5′ quencher molecules that, in the absence of a target sequence, remain annealed to form a hairpin. Binding of the probe to the target sequence disrupts the hairpin structure, leading to reporter-quencher separation and an increase in fluorescent emission. Much like multiplexed hydrolysis probe assays, described above, information on the presence of multiple specific targets can be discerned from a heterogeneous probe mixture.

### 4. Concurrent Capsid and Viral Genome Detection and Quantification Using dPCR Approach

Concurrent sample processing may be performed advantageously, where the presence of both the capsid and viral genome within a single subsample in a dPCR assay can be verified. Such concurrent sample processing may yield more accurate quantification of the active fraction than parallel sample processing. Several of the capsid and viral genome quantification methods described above can be combined into a multiplex assay, with some sample pre-processing, to enable concurrent quantification on a dPCR platform.

Non-specific capsid labeling: Non-specific protein labeling method may be used with or without a wash step, depending on the background signal of protein impurities in the sample. Non-specific protein labeling, discussed above, include, but are not limited to, fluorescent dyes like Sypro® Orange (Life Technologies) and oligonucleotides conjugated using click chemistry. While these methods will non-specifically label all proteins in the sample and, therefore, are in general not compatible with biological samples, they may offer a more cost-effective approach for homogeneous manufacturing samples.

Fluorescent capsid binding molecules: Any of the potential binding molecules previously discussed can be utilized to label the capsid prior to partitioning so long as the reporter method is able to withstand subsequent capsid dissociation conditions. Labeled capsids can be partitioned and disassembled to release the viral genome (e.g., by heat, sonication, etc.). The viral genome quantification can then be performed using any of the previously discussed methods that do not require sample pre-processing. Simultaneous to PCR-based viral genome quantification, subsamples may also be interrogated for labeled capsids. Samples positive for both viral genome and capsid readouts may be considered to have contained an intact viral capsid loaded with a genome and to be part of the active fraction. Samples positive for either the viral genome or capsid, but not both, may be considered to contain unincorporated genome or empty capsid, and can be informative on the efficiency of the manufacturing process.

Thus, included herein are methods comprising detecting both viral nucleic acid and capsid in the same partition, e.g., a partition in a plurality of partitions containing both viral nucleic acid and viral capsid component. The partition is as described herein, e.g., part of an emulsion comprising a plurality of partitions, e.g., partitions comprising aqueous phase in a continuous phase in which they are insoluble, e.g., an oil. The volume of the partition can be as described herein. The partitions may further comprise a surfactant, such as a fluorosurfactant. The continuous phase may be an oil, such as a fluorinated oil. The capsid component in the partition may comprise capsid protein, capsid lipid bilayer, or a combination thereof. Generally, the capsid component will be tagged as described elsewhere herein, e.g., tagged by binding of a component to the capsid component that is directly or indirectly detectable, also as described elsewhere herein. The method can further comprise tagging viral capsids in a bulk sample containing the virus, before the bulk sample is separated into a plurality of partitions. The method can further comprise partitioning the sample into a plurality of subsample partitions, as described herein. The plurality of partitions can also include partitions that contain capsid only and/or viral nucleic acid only, and the method can further comprise detecting capsid only in partitions containing capsid only and/or detecting viral nucleic acid only in partitions comprising viral nucleic acid only. The method can further comprise altering the integrity of the capsids in the partitions sufficiently to release viral nucleic acid, if present, while retaining the ability to manipulate the tag associated with capsid to produce or reveal a label; in certain cases, the label is already present and merely needs to be maintained. The method can further include amplifying viral nucleic acid in subsamples, e.g., partitions, if present, for example by PCR. The PCR may include amplification of viral nucleic acid using a suitable number of primers, e.g., 1, 2, 3, 4, 5, or more than 5 different sets of primers, each specific for a different sequence of the viral nucleic acid. An additional set or sets of primers may be used if capsid is tagged with an oligonucleotide to be amplified, wherein the primers are specific for the sequence of the tagging oligonucleotide. The method can further include tagging viral capsid by binding detection molecules to the surface of capsids, for example by conjugation to exposed amino acids on the surface of the capsid, or by binding to one or more specific antigens on capsid surface, such as by binding to a moiety comprising a detection molecule and a binding entity comprising an antibody, peptide, single-chain variable fragment (scFv), nanobody, aptamer, lipid, or nucleic acid. Any suitable detection molecules may be used, such as fluorescent dyes or oligonucleotides. In the latter case, detection oligonucleotides and/or viral nucleic acid, if present, are amplified in single partitions, i.e., the same partition. In certain cases, the virus can be an AAV. In such cases, the binding entity can comprise, e.g., an AAV receptor (AAVR) or fragment thereof or A20 monoclonal antibody (mAb) or fragment or aptamer thereof. In methods where a plurality of partitions are used, some of which contain viral capsid and viral nucleic acid and some of which contain only viral capsid, or only viral nucleic acid, the method can further comprise determining a ratio of number of partitions containing both viral capsid and viral nucleic acid to total number of partitions containing viral components (e.g., viral capsid and/or viral nucleic acid). In certain cases, a determination may be made as to whether viral nucleic acid is complete or partial, and partitions can be classified as containing empty capsids (no viral nucleic acid), partial capsids (viral nucleic acid but not complete), or full capsids (complete viral nucleic acid), and appropriate ratios determined (e.g., number of partitions comprising full capsids versus total number of partitions containing capsid, with or without viral nucleic acid). In certain cases, estimates of active fraction may be made, e.g., from the proportion of partitions containing full capsids. For example, partitions found to comprise both capsid and viral nucleic acid are considered part of the active fraction and partitions found to comprise either capsid or viral nucleic acid but not both are considered part of the inactive fraction.

In certain embodiments, provided herein is a method for quantifying viral genome and active fraction in a single assay comprising i) tagging viral capsids in a bulk sample containing virus; ii) partitioning the sample into a plurality of subsample partitions; iii) altering the integrity of capsids in the partitions to release viral nucleic acid, if present, while retaining the ability to detect capsid tags; and iv) detecting viral nucleic acid in partitions containing viral nucleic acid and detecting capsid in partitions containing capsid, wherein in partitions containing both viral nucleic acid and capsid, both are detected in single partitions. v) determining active fraction from the results of step (iv). Detecting viral nucleic acid can include detecting one or more sequences of viral nucleic acid that indicate functional viral nucleic acid.

In certain embodiments, provided herein is a method comprising both viral nucleic acid and capsid in single viral particles, for example by treating viral particles to tag the capsid, e.g., with a detectable label, or with a tag that can be treated to produce a detectable label, for example an oligonucleotide that is amplified and detected. The viral nucleic acid can also be amplified.

Dual target-nucleic acid binding molecules: The ‘Dual target-nucleic acid binding molecules’ method for capsid quantification easily lends itself to concurrent quantification. Following site generation or protection, and any associated wash steps, the sample can be partitioned with the novel site still bound to the capsid surface. After viral genome release, PCR methods for viral genome quantification can be optimized to include the additional capsid-associated target site using hydrolysis or hairpin probes. Subsamples may be interrogated for both viral genome and capsid, and results interpreted accordingly.

Enveloped vectors: In addition to the methods described above, labeling methods specific to enveloped vectors can also be employed. Methods previously discussed in ‘Lipid bilayer labeling methods’ can be used to label the capsid prior to partitioning. A wash step may be required to remove unbound probe. For methods in which a shift in fluorescence upon lipid bilayer insertion is observed, viral release conditions must be optimized to release the viral genome but not disassemble the capsid. Again, the viral genome quantification can be performed using any of the previously described methods that can be completed wholly within the partitions.

### 5. Kits

In certain embodiments, provided herein are kits. A kit includes a set of components, e.g., reagents and other materials, used in performing a specific function. The components are generally separately packaged, e.g., in separate containers, and the components are provided as a collection to be used together. Appropriate packaging material may also be included. Instructions, such as hard copy, electronic, or a combination thereof, may also be provided. In certain embodiments, provided herein is a kit comprising a component for tagging a viral capsid component, and primers for amplifying a section of viral nucleic acid. The component for tagging viral capsid may be specific or non-specific, depending on the needs of the end user. For example, a user who is manufacturing relatively pure transgenic viral particles may require nothing more than a non-specific tag since most or all of the material to be analyzed that can be tagged will be viral capsid; in other cases, where it is expected that non-capsid components will be present in greater quantities, the kit may include a specific tag for the viral capsid. Non-specific and specific capsid tags can be any suitable tags, such as those described herein. In certain cases, the capsid tag is specific for a component of an AAV capsid, such as AAVR or fragment thereof or A20 mAb, or fragment or aptamer thereof. The viral nucleic acid may be from any suitable virus to be analyzed; in certain embodiments, the viral nucleic acid is from AAV. The primers may be specific to a universal sequence of the AAV genome, e.g., a sequence expected to be present in an AAV regardless of whether a transgene has been introduced or not. In using such kits, the end user may provide additional primers specific to a particular transgene or set of transgenes that have potentially been added to the normal AAV genome, thus use of the kit would include use of these user-provided primers. In certain cases, the viral-capsid tagging component of the kit may comprise an oligonucleotide to be amplified, and the kit may further include appropriate primers for such amplification. The kit may further comprise a label for the tag of the viral capsid component. The kit can further comprise master mix for polymerase chain reaction.

### IV. Illustrative Combinations and Additional Examples

This section describes additional aspects and features of the methods, compositions, and kits of the present disclosure, presented without limitation as a series of paragraphs, some or all of which may be alphanumerically indexed for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

A1. A method of analyzing viral particles, the method comprising: (i) tagging capsids of the viral particles with a tag; (ii) forming subsamples of a sample containing the viral particles, each subsample of only a subset of the subsamples including at least one of the viral particles; (iii) amplifying one or more targets from a genome of the viral particles; (iv) collecting tag-related data, and amplification data for the one or more targets, from the subsamples; and (v) enumerating populations of the subsamples, the populations being defined based on the tag-related data and the amplification data.

A2. The method of paragraph A1, wherein enumerating includes obtaining subsample counts for two or more of the populations, the method further comprising determining an occupancy of the capsids by the genome using the subsample counts directly, or (uncalibrated) count values derived therefrom, in a ratio.

A3. The method of paragraph A2, wherein the count values are used in the ratio, and wherein the count values are derived from the subsample counts at least in part by normalization to scale the subsample counts and/or by adjustment based on a no particle control(s) (NPC).

A4. The method of paragraph A2 or A3, wherein tagging has a tagging efficiency, and wherein determining inherently incorporates the same tagging efficiency into a numerator and a denominator of the ratio, such that the tagging efficiency does not affect the ratio.

A5. The method of any of paragraphs A1 to A4, wherein the tag includes a template, wherein amplifying includes amplifying a first target from the template, and wherein collecting tag-related data includes collecting amplification data for the first target.

A6. The method of paragraph A5, wherein tagging includes binding copies of a pair of Proximity Assay (PA) probes including a pair of oligonucleotides to individual capsids of the viral particles, and wherein tagging also includes creating the template using the pair of oligonucleotides of the copies of the pair of PA probes for a ligation reaction or an extension reaction while the copies of the pair of PA probes remain bound to the same individual capsids.

A7. The method of paragraph A5, wherein tagging includes combining a tagging probe and the viral particles with one another, and wherein the tagging probe includes an oligonucleotide providing a preformed, full-length sequence of the template.

A8. The method of any of paragraphs A1 to A7, further comprising: combining beads and the viral particles with one another; binding the viral particles to the beads; and washing the beads after combining and before forming the subsamples.

A9. The method of any of paragraphs A1 to A8, further comprising treating the viral particles with a nuclease before tagging the capsids, and/or before forming the subsamples, to degrade nucleic acid located outside the capsids of the viral particles.

A10. The method of any of paragraphs A1 to A9, wherein tagging includes tagging a shell of the capsids, and wherein the shell forms an interior and an exterior of the capsids.

A11. The method of any of paragraphs A1 to A9, wherein tagging includes tagging an envelope of the capsids, and wherein the envelope includes a lipid bilayer.

A12. The method of any of paragraphs A1 to A11, wherein tagging includes labeling the viral particles with a label before forming the subsamples, and wherein collecting tag-related data includes illuminating the label with optical radiation and detecting light from the label.

A13. The method of any of paragraphs A1 to A12, further comprising any limitation or combination of limitations of any of paragraphs B1 to B21, C1 to C8, D1 to D19, E1 to E5, F1 to F7, G1 to G3, and H1 to H5.

B1. A method of analyzing viral particles, the method comprising: (i) tagging capsids of the viral particles with a template; (ii) forming partitions of a sample containing the viral particles, such that each partition of only a subset of the partitions includes one of the viral particles; (iii) amplifying, in the partitions, a first target from the template and one or more other targets from a genome of the viral particles; and (iv) collecting amplification data for the first target and each target of the one or more other targets from the partitions.

B2. The method of paragraph B1, further comprising enumerating populations of the partitions according to target content based on the amplification data.

B3. The method of paragraph B2, further comprising determining a capsid occupancy of the capsids by the genome using results of enumerating.

B4. The method of paragraph B3, wherein the populations include at least a pair of populations, wherein each population of the at least a pair of populations is positive for the first target, and wherein determining includes calculating a percentage or fraction of partitions in the at least a pair of populations that are also positive for each target of the one or more other targets.

B5. The method of paragraph B3 or B4, wherein enumerating includes obtaining an alpha partition count for a population of the partitions positive for the first target and negative for at least one of the one or more other targets, and a beta partition count for a population of the partitions positive for the first target and each target of the one or more other targets, and wherein determining uses values for the alpha and beta partition counts, or count values derived therefrom, to calculate a ratio representing the capsid occupancy.

B6. The method of paragraph B5, wherein the ratio includes a value for the beta partition count, or a value derived therefrom, in a numerator of the ratio, and a sum including values for the alpha and beta partition counts, or values derived therefrom, in a denominator of the ratio.

B7. The method of paragraph B5 or B6, wherein tagging has a tagging efficiency, and wherein calculating inherently incorporates the same tagging efficiency into a numerator and a denominator of the ratio, such that the tagging efficiency does not affect the ratio.

B8. The method of any of paragraphs B5 to B7, wherein the count values represent, or are derived from, normalized partition counts and/or partition counts from which no particle control (NPC) values have been subtracted.

B8a. The method of paragraph B5, wherein enumerating includes obtaining an alpha value representing the linkage concentration of partitions positive for capsids and genomes and a beta value representing a concentration of labeled capsids from which no particle control (NPC) values have been subtracted, and wherein determining uses the alpha value and beta value, or values derived therefrom, to calculate a ratio representing the capsid occupancy.

B9. The method of any of paragraphs B1 to B8a, wherein the capsids include a shell, and wherein tagging includes tagging the shell.

B10. The method of any of paragraphs B1 to B9, wherein the one or more other targets include a second target and a third target.

B11. The method of any of paragraphs B1 to B10, wherein tagging includes ligating copies of portions of the template to one another to create the template, while the copies of the portions of the template remain connected to the same individual capsids.

B12. The method of any of paragraphs B1 to B11, wherein tagging includes binding copies of a pair of Proximity Assay (PA) probes including a pair of oligonucleotides to individual capsids of the viral particles, and wherein tagging also includes creating the template using the pair of oligonucleotides for a ligation reaction or an extension reaction while the copies of the pair of PA probes remain bound to the same individual capsids.

B13. The method of paragraph B12, wherein each PA probe includes a specific binding agent that recognizes and binds to a structure of the capsids.

B14. The method of paragraph B12 or B13, wherein the PA probes are Proximity Ligation Assay (PLA) probes, and wherein tagging the capsids includes combining the pair of PLA probes with a connector such that the connector hybridizes to a 3′-end region of the oligonucleotide of one of the PLA probes and to a 5′-end region of the oligonucleotide of the other PLA probe of the pair of PLA probes.

B15. The method of any of paragraphs B1 to B10, wherein tagging includes combining a tagging probe and the viral particles with one another, and wherein the tagging probe includes a preformed, full-length sequence of the template.

B16. The method of any of paragraphs B1 to B15, wherein each partition when formed includes primers to amplify the first target and each target of the one or more other targets.

B17. The method of any of paragraphs B1 to B16, wherein the one or more other targets include a second target, wherein each partition when formed includes a first amplification probe to detect amplification of the first target and a second amplification probe to detect amplification of the second target, and wherein each amplification probe of the first and second amplification probes includes a fluorescent label.

B18. The method of any of paragraphs B1 to B17, wherein the partitions are droplets encapsulated by an immiscible continuous phase.

B19. The method of any of paragraphs B1 to B18, wherein collecting amplification data includes detecting fluorescence from the partitions.

B20. The method of any of paragraphs B1 to B19, further comprising: combining beads and the viral particles with one another; binding the viral particles to the beads; and washing the beads after combining and before tagging the capsids and/or before forming the partitions.

B21. The method of any of paragraphs B1 to B20, further comprising treating the viral particles with a nuclease to degrade nucleic acid located outside the capsids before forming the partitions.

B22. The method of any of paragraphs B1 to B21, further comprising any limitation or combination of limitations of any of paragraphs A1 to A12, C1 to C8, D1 to D19, E1 to E5, F1 to F7, G1 to G3, and H1 to H5.

C1. A composition comprising: a partition of a bulk sample containing viral particles, the partition containing only one of the viral particles; wherein a capsid of the one viral particle is tagged with a tag, and wherein the partition includes reagents sufficient for performing amplification of a target from a genome of the viral particles, if the genome is present in the partition.

C2. The composition of paragraph C1, wherein the tag includes an oligonucleotide providing a template, and wherein the partition includes reagents sufficient for performing amplification of a first target from the template and a second target from the genome, if the genome is present in the partition.

C3. The composition of paragraph C2, wherein the oligonucleotide providing the template is a single molecule attached separately to each specific binding agent of a pair of specific binding agents.

C4. The composition of paragraph C3, wherein the specific binding agents of the pair of specific binding agents are bound to the capsid separately from one another.

C5. The composition of any of paragraphs C1 to C4, wherein the reagents include an amplification probe having a label for detecting amplification of the target.

C6. The composition of any of paragraphs C1 to C5, wherein the reagents include a set of primers for amplifying the target by polymerase chain reaction (PCR).

C7. The composition of any of paragraphs C1 to C6, wherein the tag includes or is attached to a fluorophore.

C8. The composition of any of paragraphs C1 to C7, wherein the capsid contains the genome.

C9. The composition of any of paragraphs C1 to C8, further comprising any limitation or combination of limitations of any of paragraphs A1 to A12, B1 to B21, D1 to D19, E1 to E5, F1 to F7, G1 to G3, and H1 to H5.

D1. A method comprising, in a plurality of partitions containing both viral nucleic acid and viral capsid component, detecting both viral nucleic acid and capsid in the same partition.

D2. The method of paragraph D1, wherein the capsid component comprises capsid protein.

D3. The method of paragraph D1 or D2, wherein the capsid component comprises capsid lipid bilayer.

D4. The method of any of paragraphs D1 to D3, further comprising tagging viral capsids in a bulk sample containing the virus, before the bulk sample is separated into a plurality of partitions.

D5. The method of any of paragraphs D1 to D4, wherein partitions in the plurality of partitions contain capsid only and/or viral nucleic acid only, and the method further comprises detecting capsid only in partitions containing capsid only and/or detecting viral nucleic acid only in partitions comprising viral nucleic acid only.

D6. The method of any of paragraphs D1 to D5, further comprising partitioning the sample into a plurality of subsample partitions.

D7. The method of paragraph D6, further comprising altering the integrity of capsids in the subsample partitions sufficiently to release viral nucleic acid, if present, while retaining the ability to manipulate the tag associated with capsid to produce or reveal a label.

D8. The method of any of paragraphs D1 to D7, further comprising amplifying viral nucleic acid in subsamples, if present.

D9. The method of paragraph D8, wherein the viral nucleic acid is amplified by polymerase chain reaction (PCR).

D10. The method of any of paragraphs D1 to D9, wherein the viral capsids are tagged by binding detection molecules to the surface of capsids.

D11. The method of paragraph D10, wherein the binding is accomplished by conjugation to exposed amino acids on the capsid surface.

D12. The method of paragraph D10, wherein the binding is to one or more specific antigens on capsid surface.

D13. The method of paragraph D12, wherein the binding is accomplished by binding to a moiety comprising a detection molecule and a binding entity comprising an antibody, peptide, single-chain variable fragment (scFv), nanobody, aptamer, lipid, or nucleic acid.

D14. The method of any of paragraphs D1 to D13, wherein the detection molecules comprise fluorescent dyes or oligonucleotides.

D15. The method of paragraph D14, wherein the detection molecules comprise oligonucleotides.

D16. The method of paragraph D15, wherein detection oligonucleotides and/or viral nucleic acid, if present, are amplified in single partitions of the plurality of partitions.

D17. The method of any of paragraphs D1 to D16, wherein the virus is an adeno-associated virus (AAV) and the binding entity comprises an AAV receptor (AAVR) or fragment thereof or A20 monoclonal antibody (mAb) or fragment or aptamer thereof.

D18. The method of any of paragraphs D1 to D17, further comprising determining a ratio of the number of partitions that contain both viral capsid and viral nucleic acid to the number of partitions that contain viral nucleic acid or viral capsid from the results of determining the presence or absence of viral nucleic acid and/or viral capsid in the partitions.

D19. The method of paragraph D18, wherein partitions found to comprise both capsid and viral nucleic acid are considered part of the active fraction and partitions found to comprise either capsid or viral nucleic acid but not both are considered part of the inactive fraction.

D20. The method of any of paragraphs D1 to D19, further comprising any limitation or combination of limitations of any of paragraphs A1 to A12, B1 to B21, C1 to C8, E1 to E5, F1 to F7, G1 to G3, and H1 to H5.

E1. A composition comprising: i) a subsample of a larger bulk sample containing viral particles; ii) within the subsample, tagged capsid component of the virus, and reagents sufficient for performing polymerase chain reaction (PCR) on viral nucleic acid, if present.

E2. The composition of paragraph E1, further comprising viral nucleic acid.

E3. The composition of paragraph E1 or E2, wherein the reagents sufficient for performing PCR on viral nucleic acid comprises a set of primers specific for a sequence of the viral nucleic acid.

E4. The composition of any of paragraphs E1 to E3, wherein the reagents sufficient for performing PCR on viral nucleic acid comprises at least two sets of primers for two different sequences of the viral nucleic acid

E5. The composition of any of paragraphs E1 to E4, wherein the tagged capsid component is tagged with a moiety comprising an oligonucleotide, and the subsample further comprises reagents sufficient for performing PCR on the oligonucleotide of the tag.

E6. The composition of any of paragraphs E1 to E5, further comprising any limitation or combination of limitations of any of paragraphs A1 to A12, B1 to B21, C1 to C8, D1 to D19, F1 to F7, G1 to G3, and H1 to H5.

F1. A kit comprising: i) a component for tagging a viral capsid component; and ii) primers for amplifying a section of viral nucleic acid.

F2. The kit of paragraph F2, wherein the virus is an adeno-associated virus (AAV) and the primers are specific to a universal sequence in an AAV genome.

F3. The kit of paragraph F1 or F2, further comprising (iii) master mix for polymerase chain reaction.

F4. The kit of any of paragraphs F1 to F3, further comprising (iv) a label for the tag of the viral capsid component.

F5. The kit of any of paragraphs F1 to F4, wherein the component for tagging a viral capsid component is non-specific for viral capsid.

F6. The kit of any of paragraphs F1 to F5, wherein the component for tagging a viral capsid component is specific for viral capsid.

F7. The kit of any of paragraphs F1 to F6, wherein the viral capsid comprises capsid from an AAV and the component specific for viral capsid comprises AAVR or a fragment thereof, or A20 monoclonal antibody (mAb) or fragment or aptamer thereof.

F8. The kit of any of paragraphs F1 to F7, further comprising any limitation or combination of limitations of any of paragraphs A1 to A12, B1 to B21, C1 to C8, D1 to D19, E1 to E5, G1 to G3, and H1 to H5.

G1. A method for quantifying viral genome and active fraction in a single assay, the method comprising: i) tagging viral capsids in a bulk sample containing virus; ii) partitioning the sample into a plurality of subsample partitions; iii) altering the integrity of capsids in the partitions to release viral nucleic acid, if present, while retaining the ability to detect capsid tags; and iv) detecting viral nucleic acid in partitions containing viral nucleic acid and detecting capsid in partitions containing capsid, wherein in partitions containing both viral nucleic acid and capsid, both are detected in single partitions.

G2. The method of paragraph G1, further comprising (v) determining active fraction from the results of step (iv).

G3. The method of paragraph G1 or G2, wherein detecting viral nucleic acid comprises detecting one or more sequences of viral nucleic acid that indicate functional viral nucleic acid.

G4. The method of any of paragraphs G1 to G3, further comprising any limitation or combination of limitations of any of paragraphs A1 to A12, B1 to B21, C1 to C8, D1 to D19, E1 to E5, F1 to F7, and H1 to H5.

H1. A method comprising detecting both viral nucleic acid and capsid in single viral particles.

H2. The method of paragraph H1, further comprising treating the viral particles to tag the viral capsid.

H3. The method of paragraph H1 or H2, further comprising labeling the tagged viral capsid with a detectable label.

H4. The method of paragraph H3, wherein the label is part of the tag.

H5. The method of any of paragraphs H1 to H4, wherein the viral nucleic acid is amplified.

H6. The method of any of paragraphs H1 to H5, further comprising any limitation or combination of limitations of any of paragraphs A1 to A12, B1 to B21, C1 to C8, D1 to D19, E1 to E5, F1 to F7, and G1 to G3.

### V. Advantages and Benefits

The different examples of methods, compositions, and kits for analysis of viral particles provide several advantages over known solutions for analyzing viral particles. For example, illustrative examples described herein permit calibration-free quantification of capsid occupancy for a set of viral particles. Without the need for a separate calibration for the efficiency of capsid detection, viral particles can be analyzed faster, with less expense, higher accuracy, and/or reduced labor.

Additionally, the methods, compositions, and kits disclosed herein allow measurement of viral titer and capsid occupancy in the same assay.

No known system or method can perform these functions. However, not all examples described herein provide the same advantages or the same degree of advantage.

### VI. Conclusion

The disclosure set forth above may encompass multiple distinct examples with independent utility. Although each of these has been disclosed in its preferred form(s), the specific examples thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed:

1. A method of analyzing viral particles, the method comprising:
tagging capsids of the viral particles with a tag;
forming subsamples of a sample containing the viral particles, each subsample of only a subset of the subsamples including at least one of the viral particles;
amplifying one or more targets from a genome of the viral particles;
collecting tag-related data, and amplification data for the one or more targets, from the subsamples; and
enumerating populations of the subsamples, the populations being defined based on the tag-related data and the amplification data.

2. The method of claim 1, wherein enumerating includes obtaining subsample counts for two or more of the populations, the method further comprising determining an occupancy of the capsids by the genome using the subsample counts directly, or count values derived therefrom, in a ratio.

3. The method of claim 2, wherein the count values are used in the ratio, and wherein the count values are derived from the subsample counts at least in part by normalization to scale the subsample counts and/or by adjustment based on a no particle control(s) (NPC).

4. The method of claim 2, wherein tagging has a tagging efficiency, and wherein determining inherently incorporates the same tagging efficiency into a numerator and a denominator of the ratio, such that the tagging efficiency does not affect the ratio.

5. The method of claim 1, wherein the tag includes a template, wherein amplifying includes amplifying a first target from the template, and wherein collecting tag-related data includes collecting amplification data for the first target.

6. The method of claim 5, wherein tagging includes binding copies of a pair of Proximity Assay (PA) probes including a pair of oligonucleotides to individual capsids of the viral particles, and wherein tagging also includes creating the template using the pair of oligonucleotides of the copies of the pair of PA probes for a ligation reaction or an extension reaction while the copies of the pair of PA probes remain bound to the same individual capsids.

7. The method of claim 1, further comprising:
combining beads and the viral particles with one another;
binding the viral particles to the beads; and
washing the beads after combining and before forming the subsamples.

8. A method of analyzing viral particles, the method comprising:
tagging capsids of the viral particles with a template;
forming partitions of a sample containing the viral particles, such that each partition of only a subset of the partitions includes one of the viral particles;
amplifying, in the partitions, a first target from the template and one or more other targets from a genome of the viral particles; and
collecting amplification data for the first target and each target of the one or more other targets from the partitions.

9. The method of claim 8, further comprising:
enumerating populations of the partitions according to target content based on the amplification data.

10. The method of claim 9, further comprising:
determining a capsid occupancy of the capsids by the genome using results of enumerating.

11. The method of claim 10, wherein the populations include at least a pair of populations, wherein each population of the at least a pair of populations is positive for the first target, and wherein determining includes calculating a percentage or fraction of partitions in the at least a pair of populations that are also positive for each target of the one or more other targets.

12. The method of claim 10, wherein enumerating includes obtaining an alpha partition count for a population of the partitions positive for the first target and negative for at least one of the one or more other targets, and a beta partition count for a population of the partitions positive for the first target and each target of the one or more other targets, and wherein determining uses values for the alpha and beta partition counts, or count values derived therefrom, to calculate a ratio representing the capsid occupancy.

13. The method of claim 12, wherein the ratio includes a value for the beta partition count, or a value derived therefrom, in a numerator of the ratio, and a sum including values for the alpha and beta partition counts, or values derived therefrom, in a denominator of the ratio.

14. The method of claim 12, wherein tagging has a tagging efficiency, and wherein calculating inherently incorporates the same tagging efficiency into a numerator and a denominator of the ratio, such that the tagging efficiency does not affect the ratio.

15. The method of claim 12, wherein the count values represent, or are derived from, normalized partition counts and/or partition counts from which no particle control (NPC) values have been subtracted.

16. The method of claim 10, wherein enumerating includes obtaining an alpha value representing the linkage concentration of partitions positive for capsids and genomes and a beta value representing a concentration of labeled capsids from which no particle control (NPC) values have been subtracted, and wherein determining uses the alpha value and beta value, or values derived therefrom, to calculate a ratio representing the capsid occupancy.

17. The method of claim 8, wherein tagging includes ligating copies of portions of the template to one another to create the template, while the copies of the portions of the template remain connected to the same individual capsids.

18. The method of claim 8, wherein tagging includes binding copies of a pair of Proximity Assay (PA) probes including a pair of oligonucleotides to individual capsids of the viral particles, and wherein tagging also includes creating the template using the pair of oligonucleotides for a ligation reaction or an extension reaction while the copies of the pair of PA probes remain bound to the same individual capsids.

19. The method of claim 8, further comprising:
combining beads and the viral particles with one another;
binding the viral particles to the beads; and
washing the beads after combining and before forming the partitions.

20. A composition comprising:
a partition of a bulk sample containing viral particles, the partition containing only one of the viral particles;
wherein a capsid of the one viral particle is tagged with a tag, and wherein the partition includes reagents sufficient for performing amplification of a target from a genome of the viral particles, if the genome is present in the partition.
